(19) United States
(12) Patent Application Publication
(10) Pub. No.: US 2026/0191634 A1
(43) Pub. Date: Jul. 9, 2026

(54) REDUCING CHARGE LEAKAGE FROM A PIEZOELECTRIC VALVE IN A UROLOGY IMPLANTABLE MEDICAL DEVICE

(71) Applicant: Boston Scientific Scimed, Inc., Maple Grove, MN (US)

(72) Inventors: James Stephen Coughlin, Shoreview, MN (US); Brian Thomas Swanson, Fridley, MN (US); Kevin James Ivanca, Minneapolis, MN (US); Keith R. Maile, New Brighton, MN (US); Jotham David Bremseth, St. Paul, MN (US)

(21) Appl. No.: 19/440,911

(22) Filed: Jan. 6, 2026

Related U.S. Application Data
(60) Provisional application No. 63/743,060, filed on Jan. 8, 2025.

(51) Int. Cl. A61F 2/00; A61F 2/26; A61F 2/48

(52) U.S. Cl. CPC A61F 2/004; A61F 2/26; A61F 2/482; A61F 2/484; A61F 2250/0003; A61F 2250/0013

(57) ABSTRACT

The techniques described herein relate to an implantable fluid-operated device configured to control fluid flow between a fluid reservoir and an inflatable member. The device includes: a battery configured for storing energy, energy transmission circuitry configured for receiving energy from an external energy transmission device and providing energy to charge the battery, and a valve. The valve includes a base plate, a deformable diaphragm, a piezoelectric element coupled to the deformable diaphragm, and a fluid chamber defined between the base plate and the deformable diaphragm. The device further includes a valve driver configured for providing a voltage to the piezoelectric element to change a state of the valve between an open state and a closed state and a high impedance switch configured for switching the voltage from the valve driver to the piezoelectric element.

### CROSS-REFERENCE TO RELATED APPLICATION

This application claims priority to U.S. Provisional Patent Application No. 63/743,060, filed on January 8, 2025, entitled “REDUCING CHARGE LEAKAGE FROM A PIEZOELECTRIC VALVE IN A UROLOGY IMPLANTABLE MEDICAL DEVICE”, the disclosure of which is incorporated by reference herein in its entirety.

### TECHNICAL FIELD

This disclosure relates generally to bodily implants and, in particular, to reducing charge leakage from a piezoelectric valve in a urology implantable medical device.

### BACKGROUND

Active implantable fluid-operated inflatable devices can include one or more pumps that regulate the flow of fluid between different portions of the implantable device. One or more valves can be positioned within fluid passageways of the device to direct and control the flow of fluid to achieve inflation, deflation, pressurization, depressurization, activation, deactivation and the like of different fluid-filled components of the device. In some implantable fluid-operated devices, an implantable pumping device may be manually operated by the user to provide for the transfer of fluid between a reservoir and the fluid-filled implant components of the device. In some situations, manual operation of the pumping device may make it difficult to achieve consistent inflation, deflation, pressurization, depressurization, activation, deactivation and the like of the fluid-filled implant components. Inconsistent inflation, deflation, pressurization, depressurization, activation and/or deactivation of the fluid-filled implant device(s) may adversely affect patient comfort, efficacy of the device, and the overall patient experience. Some implantable fluid-operated devices include an electronic control system including an electronically controlled manifold providing for the transfer of fluid within the implantable fluid-operated device.

The use of the electronic control system may provide for more accurate actuation and control of the flow of fluid between components of the inflatable device, thus improving performance and efficacy of the device, as well as patient comfort and safety. The electronic control system may include one or more electronically-operated pumps and one or more valves to control the flow of fluid in the system, and the pumps and valves may be operated by way of piezoelectric elements associated with the pumps and valves. During operation, it may be necessary for one or more electronically-operated valves to remain open or closed for an extended period of time, and this may require maintaining the one or more valves in a particular electrical state. However, over time, the electrical charge on the valves may decay, such that the valves are not maintained in their open or closed states.

Thus, techniques for maintaining valves in an open or closed state for an extended period of time are needed.

### SUMMARY

In a general aspect, the techniques described herein relate to an implantable fluid-operated device configured to control fluid flow between a fluid reservoir and an inflatable member, the device including: a battery configured for storing energy; energy transmission circuitry configured for receiving energy from an external energy transmission device and providing energy to charge the battery; a valve including: a base plate, a deformable diaphragm, a piezoelectric element coupled to the deformable diaphragm, and a fluid chamber defined between the base plate and the deformable diaphragm; a valve driver configured for providing a voltage to the piezoelectric element to change a state of the valve between an open state and a closed state; and a high impedance switch configured for switching the voltage from the valve driver to the piezoelectric element.

Implementations can include one or more of the following features, alone or in any combination with each other.

For example, the high impedance switch can include a solid state relay.

In another example, the high impedance switch can include a bidirectional, multi-FET circuit.

In another example, the valve driver can include voltage sensing circuitry configured for measuring a voltage across the piezoelectric element when the high impedance switch is connected between the valve driver and the valve.

In another example, the techniques described herein relate to an implantable fluid-operated device, wherein the inflatable member can include an inflatable cuff configured for implantation about a urethra of a patient.

In another example, the techniques described herein relate to an implantable fluid-operated device, wherein the inflatable member can include a penile implant.

In another example, the device can further include a controller, the controller being configured to: close the switch; cause the valve driver to provide a predetermined voltage to the piezoelectric element when the switch is closed; open the switch; cause the valve driver to measure a voltage on the piezoelectric element when the switch is closed; and cause the valve driver to provide the predetermined voltage to the piezoelectric element when the measured voltage is less than a threshold voltage.

In another example, the device can further include a controller, the controller being configured to: close the switch; cause the valve driver to provide a predetermined voltage to the piezoelectric element when the switch is closed; open the switch; repeatedly, when the switch is open, determine whether a time after the opening of the switch exceeds a threshold time; and when the time exceeds the threshold time: close the switch; cause the valve driver to add charge to the piezoelectric element to provide the predetermined voltage to the piezoelectric element; and open the switch.

In another example, the device can further include a controller and an isolation circuit connected between the controller and the piezoelectric element and being configured to providing a voltage signal proportional to a voltage across the piezoelectric element, where the controller configured to: close the switch; cause the valve driver to provide a predetermined voltage to the piezoelectric element when the switch is closed; open the switch; when the switch is open, determine a voltage across the piezoelectric element based on the voltage signal; and cause the valve driver to provide the predetermined voltage to the piezoelectric element when the determined voltage is less than a threshold voltage.

In another example, the isolation circuit can include a buffer.

In another example, the isolation circuit can include an instrumentation amplifier.

In another example, the device can further include a pressure sensor configured for measuring a pressure in the inflatable member and a controller configured to, in response to a pressure measured by the pressure sensor being less than a threshold pressure: close the switch; and cause the valve driver to provide a predetermined voltage to the piezoelectric element.

In another general aspect, the techniques described herein relate to a method of controlling fluid flow between a fluid reservoir and an inflatable member of an implantable fluid-operated device, the method including: receiving energy from an external energy transmission device; charging a battery of the implantable fluid-operated device with the received energy; providing, with a valve driver, a voltage from the battery to a piezoelectric element of a piezoelectric valve that is fluidically coupled between the fluid reservoir and the inflatable member to change a state of the valve between an open state and a closed state; after providing the voltage to the piezoelectric element, opening a high impedance switch between the valve driver to the piezoelectric element.

Implementations can include one or more of the following features, alone or in any combination with each other.

In another example, the inflatable member can include an inflatable cuff configured for implantation about a urethra of a patient.

In another example, the inflatable member can include a penile implant.

In another example, the method can further include: closing the switch before the voltage is provided to the piezoelectric element; providing, with the valve driver, a predetermined voltage to the piezoelectric element when the switch is closed; after providing the predetermined voltage to the piezoelectric element, opening the switch; when the switch is open, repeatedly determining whether a time after the opening of the switch exceeds a threshold time; and when the time exceeds the threshold time: closing the switch; adding charge from the battery to the piezoelectric element to provide the predetermined voltage to the piezoelectric element; and opening the switch.

In another example, the method can further include: generating, with an isolation circuit that is connected between a controller and the piezoelectric element, a voltage signal proportional to a voltage across the piezoelectric element; when the switch is open, determining a voltage across the piezoelectric element based on the voltage signal; providing a predetermined voltage to the piezoelectric element when the determined voltage is less than a threshold voltage.

In another example, the isolation circuit can include a buffer.

In another example, the isolation circuit can include an instrumentation amplifier

In another example, the method can further include: measuring a pressure in the inflatable member; and, in response to the measured pressure being less than a threshold pressure: closing the switch; and providing a predetermined voltage to the piezoelectric element.

### BRIEF DESCRIPTION OF THE DRAWINGS

FIG. 1 is a block diagram of an implantable fluid-operated inflatable device.

FIG. 2A illustrates a system including an example implantable fluid-operated inflatable device.

FIG. 2B illustrates a system including another example implantable fluid-operated inflatable device.

FIG. 3 is a schematic diagram of a fluidic architecture of an implantable fluid-operated inflatable device.

FIG. 4A is an exploded view of an example valve device of a fluid control system of a fluid-operated inflatable device.

FIG. 4B is another exploded view of the example valve device shown in FIG. 4A.

FIG. 4C is a cross-sectional view of the example valve device shown in FIG. 4A, in a closed position.

FIG. 4D is a cross-sectional view of the example valve device shown in FIG. 4A, in an open position.

FIG. 5A is a schematic view of an example valve device including an example auxiliary flow control device, with the example valve device in an open position.

FIG. 5B is a schematic view of an example valve device including an example auxiliary flow control device, with the example valve device in a closed position.

FIG. 6A is an exploded view of an example pump device of a fluid control system of a fluid-operated inflatable device.

FIG. 6B is a cross-sectional view of the example pump device shown in FIG. 6A, in an open position.

FIGS. 7A, 7B, and 7C are cross-sectional views of example pump devices that includes a filter for capturing particulate matter in the fluid flow and/or for blocking the particulate matter from entering certain parts of the fluidic system (e.g., for blocking particulate matter from entering a pump chamber of the device).

FIG. 8 is a schematic end view of a filter foil.

FIGS. 9A, 9B, and 9C are cross-sectional views of example pump devices that includes a filter for capturing particulate matter in the fluid flow and/or for blocking the particulate matter from entering certain parts of the fluidic system (e.g., for blocking particulate matter from entering a pump chamber of the device).

FIG. 10 is a cross-sectional view of the valve device of FIG. 5A and 5B, but also including a filter located at an end of a second fluid passageway and a filter located within a first fluid passageway.

FIG. 11 is a schematic block diagram of a system for driving a piezoelectric element of a piezoelectric-operated pump or valve and for monitoring and controlling the performance of the piezoelectric element.

FIG. 12 is a schematic diagram of an implantable fluid-operated inflatable device that reduces leakage current from piezoelectric valves of the device.

FIG. 13 is a schematic diagram of a system including components of an implantable fluid-operated inflatable device used to operate a piezoelectric valve of the device.

FIGS. 14A and 14B are schematic diagrams of processes for controlling a system to close a piezoelectric valve and to maintain the piezoelectric valve in its closed state.

FIG. 15 is a schematic diagram of a system including components of an implantable fluid-operated inflatable device used to operate a piezoelectric valve of the device.

FIG. 16 is a flowchart of an example process for controlling fluid flow between a fluid reservoir and an inflatable member of an implantable fluid-operated device.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having,” as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

An implantable fluid-operated inflatable device may include a fluid control system. In some examples, the fluid control system includes at least one pump and/or at least one valve. In some examples, the components of the fluid control system control the flow of fluid between a fluid reservoir and an inflatable member of the implantable fluid-operated inflatable device, to provide for the inflation/pressurization and deflation/depressurization of the inflatable member. In some implementations, the fluid control system can be electronically-operated.

For example, the pumps and/or valves of the fluid control system can be electronically-operated by the fluid control system to control the pressure of, and the flow of fluid in, parts of the fluid-operated inflatable device. An electronically-operated fluid control system, in accordance with implementations described herein, can include a plurality of electromechanical devices, such as piezoelectric devices that operate as pumps or as valves in the system. One or more controllers can control the electromechanical devices. Additionally, the one or more controllers can monitor the performance and electrical properties of the electromechanical devices to detect errors, failures, and degradation of the devices. When an error, failure, or degradation of an electromechanical device is detected, the one or more controllers can adjust the electronic control of the electromechanical device to facilitate continued operation of the electromechanical device and the safety of the patient in whom the inflatable device is implanted.

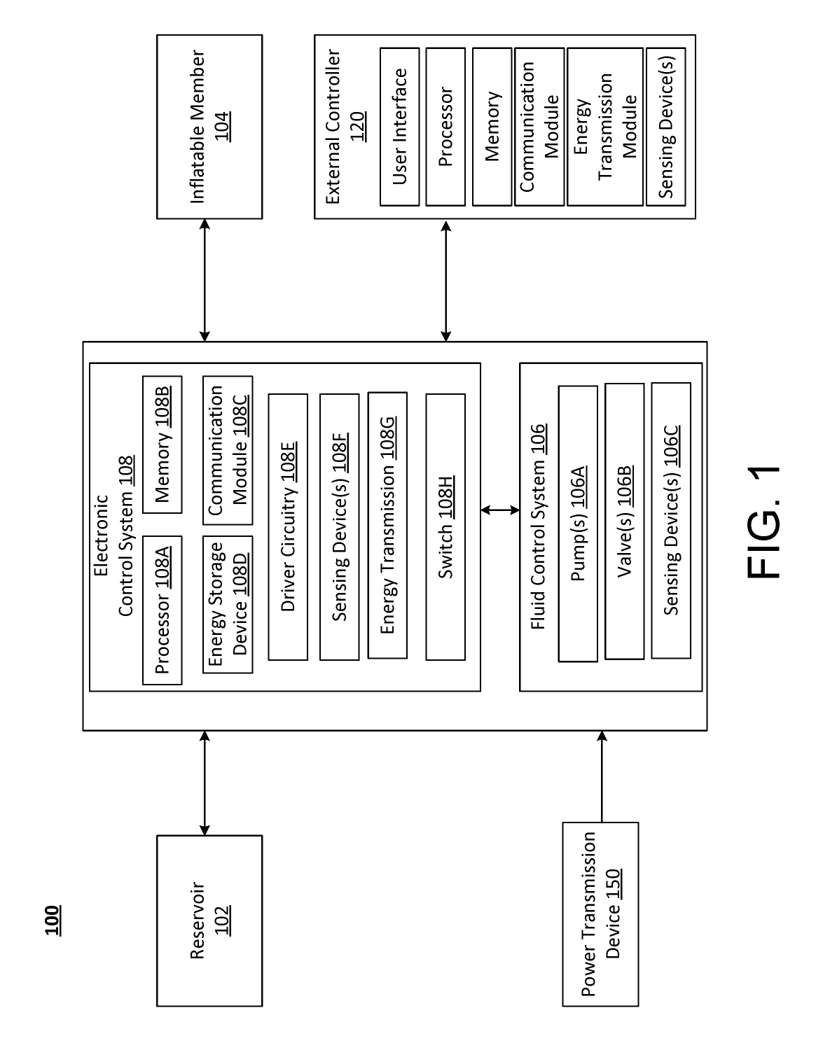

FIG. 1 is a block diagram of an example implantable fluid-operated inflatable device 100. The example inflatable device 100 shown in FIG. 1 includes a fluid reservoir 102, an inflatable member 104, and an electronic control system 108. The electronic control system 108 may interface with a fluid control system 106. The fluid control system 106 can include fluidics components such as one or more pumps 106A, one or more valves 106B and the like configured to transfer fluid between the fluid reservoir 102 and the inflatable member 104. The fluid control system 106 can include one or more sensing devices 106C, such as, for example, one or more pressure sensors, one or more flow rate sensors, etc., that sense conditions such as, for example, fluid pressure, fluid flow rate and the like within the fluidics architecture of the inflatable device 100. In some implementations, the electronic control system 108 includes components that provide for the monitoring and/or control of the operation of various fluidics components of the fluid control system 106 and/or communication with one or more sensing device(s) within the implantable fluid-operated inflatable device 100 and/or communication with one or more external device(s). In some examples, the electronic control system 108 includes components such as a processor 108A, a memory 108B, a communication module 108C, an energy storage device 108D (e.g., a battery), electronic driver circuity 108E, sensing devices 108F, such as, for example, voltage measurement circuitry, current measurement circuitry, an accelerometer, one or more switches 108H, and other such components configured to provide for the monitoring, operation, and control of the implantable fluid-operated inflatable device 100, and energy transmission circuitry 108G. In some examples, the communication module 108C of the electronic control system 108 may provide for communication with one or more external devices such as, for example, an external controller 120.

In some examples, the external controller 120 includes components such as, for example, a user interface, a processor, a memory, a communication module, an energy transmission module, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface, and to transmit the user inputs, for example, via the communication module, to the electronic control system 108 for processing, operation, and control of the inflatable device 100. Similarly, the electronic control system 108 may, via the respective communication modules, transmit operational information to the external controller 120. This may allow operational status of the inflatable device 100 to be provided, for example, through the user interface of the external controller 120, to the user, may allow diagnostics information to be provided to a physician, a technician, and the like.

In some examples, the energy transmission module of the external controller 120 provides for charging of the components of the internal electronic control system 108. In some examples, transmission of energy for the charging of the internal electronic control system 108 can be, alternatively or additionally, provided by an external energy transmission device 150 that is separate from the external controller 120. In some implementations the external controller 120 can include sensing devices such as one or more pressure sensors, one or more accelerometers, and other such sensing devices. In some implementations, a pressure sensor in the external controller 120 may provide, for example, a local atmospheric or working pressure to the internal electronic control system 108, to allow the inflatable device 100 to compensate for variations in pressure. In some implementations, an accelerometer in the external controller 120 may provide detected patient movement to the internal electronic control system 108 for control of the inflatable device 100.

The fluid reservoir 102, the inflatable member 104, the electronic control system 108 and the fluid control system 106 may be internally implanted into the body of the patient. In some implementations, the electronic control system 108 and the fluid control system 106 are coupled in, or incorporated into, a housing. In some implementations, at least a portion of the electronic control system 108 is physically separate from the fluid control system 106. In some implementations, some modules of the electronic control system 108 are coupled to, or incorporated into, the fluid control system 106, and some modules of the electronic control system 108 are separate from the fluid control system 106. For example, in some implementations, some modules of the electronic control system 108 are included in an external device (such as the external controller 120) that is in communication with other modules of the electronic control system 108 included within the implantable fluid-operated inflatable device 100.

In some examples, electronic monitoring and control of the implantable fluid-operated inflatable device 100 may provide for improved patient control of the device, improved patient comfort, improved patient safety, and the like. In some examples, electronic monitoring and control of the implantable fluid-operated inflatable device 100 may afford the opportunity for tailoring of the operation of the inflatable device 100 by a physician without further surgical intervention. The fluidic architecture defining the flow and control of fluid through the implantable fluid-operated inflatable device 100, including the configuration and placement of fluidics components such as pumps, valves, sensing devices and the like, may allow the inflatable device 100 to precisely monitor and control operation of the inflatable device, effectively respond to user inputs, and quickly and effectively adapt to changing conditions both within the inflatable device 100 (changes in pressure, flow rate and the like) and external to the inflatable device 100 (pressure surges due to physical activity, impacts and the like, sustained pressure changes due to changes in atmospheric conditions, and other such changes in external conditions).

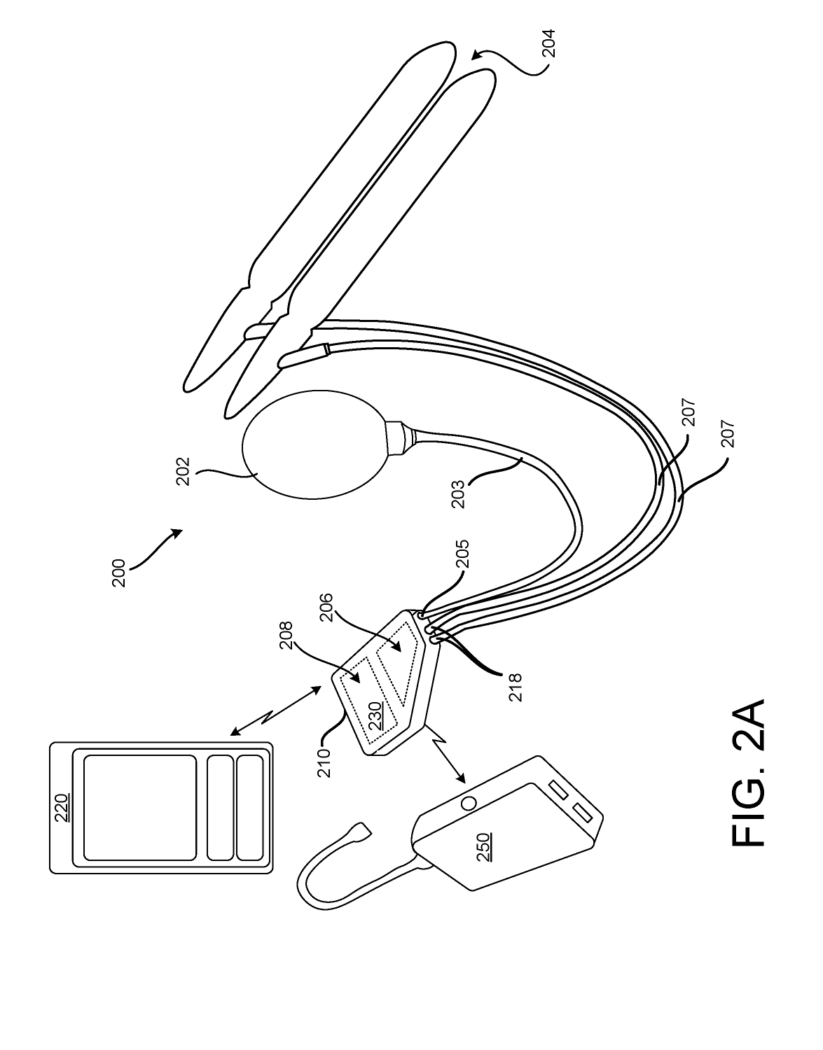

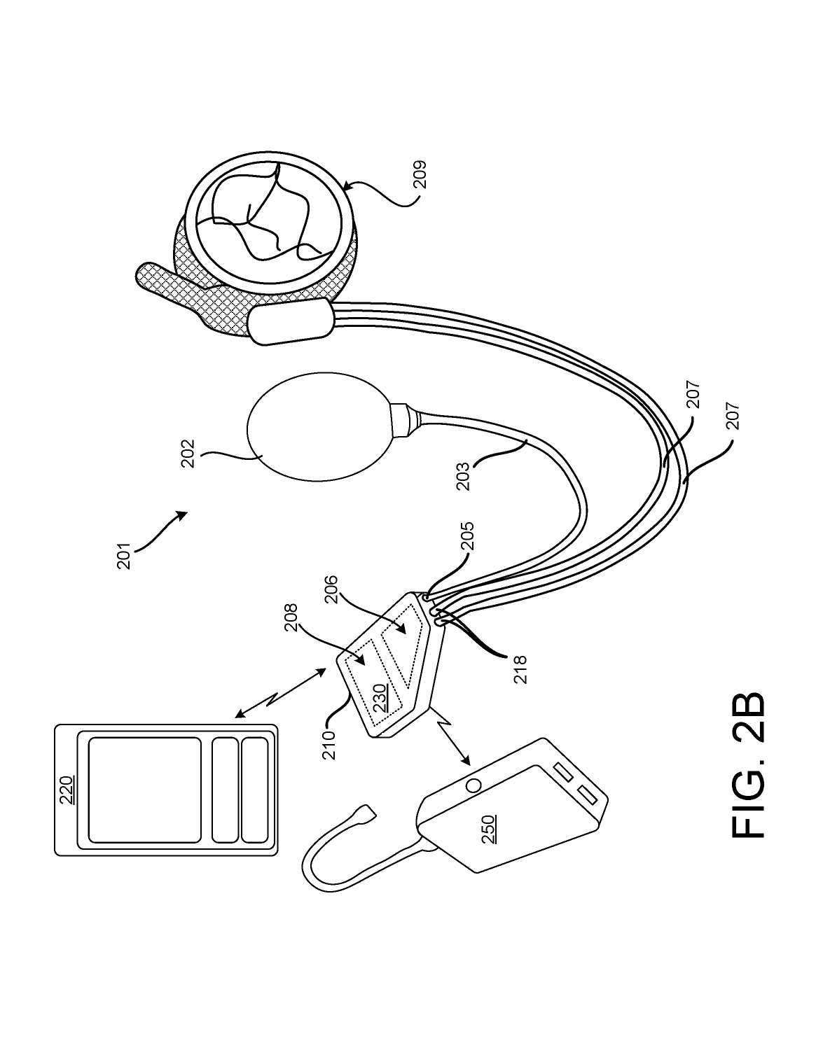

The example implantable fluid-operated inflatable device 100 may be representative of a number of different types of implantable fluid-operated devices. For example, the implantable fluid-operated inflatable device 100 shown in FIG. 1 may be representative of an inflatable penile prosthesis as shown in FIG. 2A or an inflatable artificial urinary tract sphincter as shown in FIG. 2B. In some implementations, the example implantable fluid-operated inflatable device 100 shown in FIG. 1 may be representative of other types of implantable inflatable devices that rely on the control of fluid flow to components of the device to achieve inflation, pressurization, deflation, depressurization, deactivation, and the like, such as, for example, an artificial urinary sphincter, and other such devices.

An example system including an example implantable fluid-operated inflatable device 200 in the form of an example inflatable penile prosthesis is shown in FIG. 2A. Another example system including an example implantable fluid-operated inflatable device 201 in the form of an example artificial urinary tract sphincter is shown in FIG. 2B. The example implantable fluid-operated inflatable device 200 includes a fluid control system 206 (similar to the example fluid control system 106 described above with respect to FIG. 1) including fluidics components such as pumps, valves, sensing devices and the like positioned in fluid passageways. In some implementations, the fluid control system includes components such as, for example, one or more fluid control devices, one or more pressure sensors, and other such components. In some implementations, the example implantable fluid-operated inflatable device 200 includes an electronic control system 208 (similar to the example electronic control system 108 described above with respect to FIG. 1) configured to provide for the transfer of fluid between a reservoir 202 (such as the example fluid reservoir 102 described above with respect to FIG. 1) and an inflatable member 204 (similar to the example inflatable member 104 described above with respect to FIG. 1) via the fluidics components. In the example shown in FIG. 2A, the inflatable member 204 is in the form of a pair of inflatable cylinders, which are configured for implantation within the penis of a patient. In the example shown in FIG. 2B, the inflatable member 209 is in the form of an inflatable cuff that is configured for implantation around the urethra of a patient. In the examples shown in FIGS. 2A and 2B, fluidics components of the fluid control system 206, and electronic components of the electronic control system 208 are received in a housing 210. In some implementations, fluidics components of the fluid control system 206, and electronic components of the electronic control system 208 received in the housing 210 together define an electronically controlled fluid manifold 230 that provides for the electronic control of the flow of fluid between the reservoir 202 and the inflatable member 204 or the inflatable member 209.

In the example shown in FIG. 2A, a first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 218 of the electronically controlled fluid manifold 230 (the fluid control system 206/electronic control system 208 received in the housing 210) with the inflatable member 204 in the form of the inflatable cylinders. In some examples, the electronic control system 208 can communicate with an external controller 220 (similar to the external controller 120 described above with respect to FIG. 1), via respective communication modules. For example, an application stored in a memory and executed by a processor of the external controller 220 may allow the user and/or a physician to operate, view, monitor and alter operation of the implantable fluid-operated inflatable device 200. In some examples, components of the electronic control system 208 and/or the fluid control system 206 can be charged and/or recharged by an energy transmission module of the external controller 220, and/or by an energy transmission device 250, that is separate from the external controller 220. The example implantable fluid-operated inflatable device 200 shown in FIG. 2A includes an electronic control system 208 to provide for control of the operation of the respective inflatable members 204 in the form of cylinders, and the monitoring and control of pressure and/or fluid flow through inflatable members 204.

The principles to be described herein are applicable to the example implantable fluid-operated inflatable device, in the form of the example inflatable penile prostheses shown in FIG. 2A, and to other types of implantable fluid-operated inflatable devices that rely on pumps, valves and/or various fluidics components to provide for the transfer of fluid between the different fluid-filled implantable components to achieve inflation, deflation, pressurization, depressurization, deactivation, occlusion, and the like for effective operation. For example, as shown in FIG. 2B, the inflatable member 209 can include an inflatable cuff, which may be implemented as an artificial urinary sphincter. The inflatable cuff 209 is or may be disposed about a urethra proximate the bladder. The implantable fluid-operated inflatable device 201 can be activated to pump fluid from a reservoir to expand the cuff 209 and to close the urethra. The cuff 209 is deflated to allow a patient to void the bladder.

As noted above, the electronic control system 208 controlling the flow of fluid between the reservoir 202 and the inflatable member 204 for inflation, pressurization, deflation, depressurization and the like of the inflatable member 204 may provide for improved patient control of the implantable fluid-operated inflatable device 200, improved accuracy in operation of the implantable fluid-operated inflatable device 200, improved patient comfort, improved patient safety, and the like. In some situations, this improved control and improved accuracy in the operation of the implantable fluid-operated inflatable device 200 may rely on precise operation and control of the components within the fluid control system 206 and/or the electronically controlled fluid manifold 230. Accordingly, in some implementations, the electronically controlled fluid manifold 230 includes a fluid control system 206 having one or more pump and/or one or more valve devices. Accurate and consistent operation of the components of the pump and/or valve devices may produce the desired accurate flow control, and consistent inflation, deflation, pressurization, depressurization, deactivation, occlusion, and the like for effective operation.

A fluid control system, in accordance with implementations described herein, can include a pump assembly including, for example, one or more pump devices and valve devices within a fluid circuit of the pump assembly to control the transfer fluid between the fluid reservoir and the inflatable member. In some examples, the pump assembly including the one or more pump devices and valve device(s) is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump devices and valve devices include electric elements that are configured to be electronically actuated to change their shape and thereby to function as a pump or valve. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization, and deactivation of the components of the implantable fluid-operated device to provide for patient safety and device efficacy.

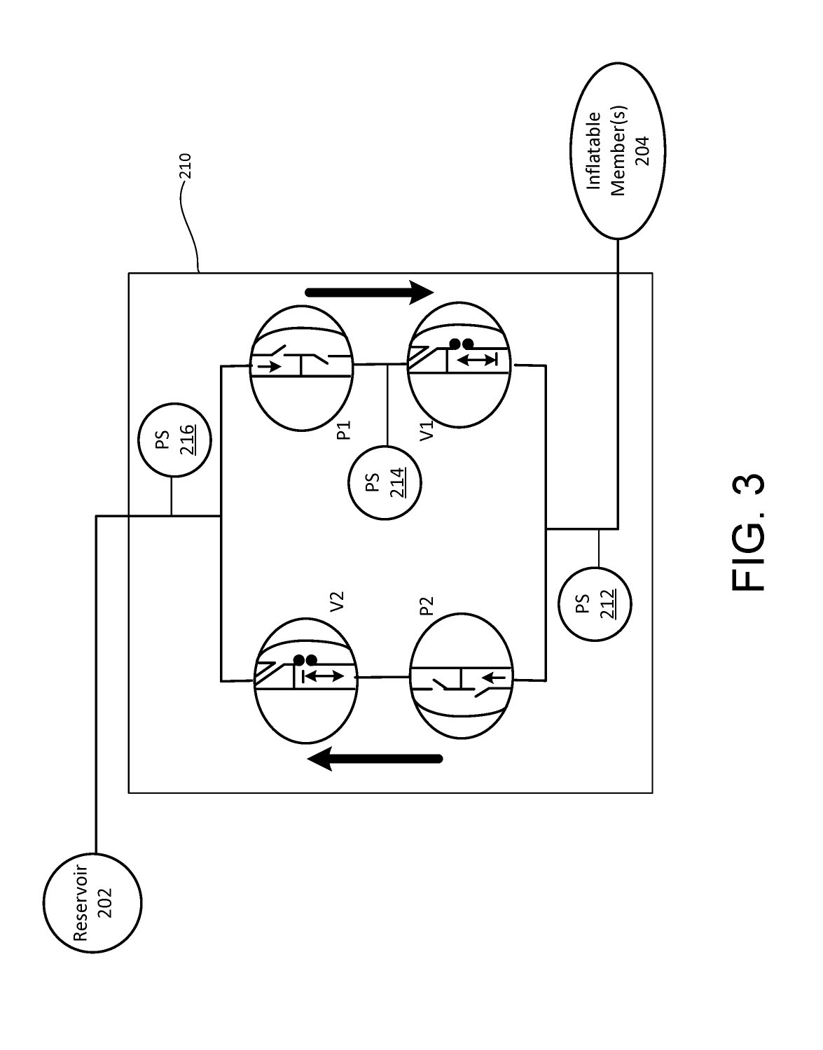

FIG. 3 is a schematic diagram of an example fluidic architecture for an electronically-operated implantable fluid-operated inflatable device, according to an aspect. The fluidic architecture of an implantable fluid-operated inflatable device can include other arrangements of fluidic passageways, pump(s)/valve(s), pressure sensor(s) and other components than the examples shown in FIG. 3.

The example fluidic architecture shown in FIG. 3 includes a first pump P1 and a first valve V1 positioned in a first fluid passageway, between the reservoir 202 and the inflatable member 204, to control the flow of fluid from the reservoir 202 to the inflatable member 204. The example fluidic architecture shown in FIG. 3 includes a second pump P2 and a second valve V2 positioned in a second fluid passageway, between the inflatable member 204 and the reservoir 202, to control the flow of fluid from the inflatable member 204 to the reservoir 202.

In example fluidic architecture shown in FIG. 3, the first pump P1 and the first valve V1 operate to pump fluid from the reservoir 202 to the inflatable member 204 through the first fluid passageway to provide for inflation of the inflatable member 204, while the second valve V2 closes the second fluid passageway to prevent backflow of fluid, back to the reservoir 202. The second pump P2 and the second valve V2 operate to pump fluid from the inflatable member 204 to the reservoir 202 through the second fluid passageway to provide for deflation of the inflatable member 204, while the first valve V1 closes the first fluid passageway to prevent backflow of fluid to the inflatable member 204.

In some implementations, the example fluidic architecture can include one or more pressure sensors 212, 214, 216, each configured to measure a fluid pressure at a point in the system. For example, a first pressure sensor 212 can be connected to a fluidic passageway, conduit, chamber or component located fluidically between the inflatable member 204 and pumps P1, P2 and valves V1, V2, and can be configured to measure a fluid pressure at this location, which can also serve as a measure of a fluid pressure in the inflatable member(s) 204, because the fluid is essentially incompressible and the conduit between the pressure sensor 212 and the inflatable member(s) 204 can be considered to be free of obstruction. A second pressure sensor 214 can be connected to a fluidic passageway, conduit, chamber or component located fluidically between pump P1 and valve V1 and can be configured to measure a fluid pressure at this location. A third pressure sensor 216 can be connected to a fluidic passageway, conduit, chamber or component located fluidically between the reservoir 202 and pumps P1, P2 and valves V1, V2, and can be configured to measure a fluid pressure at this location, which can also serve as a measure of a fluid pressure in the reservoir, because the fluid is essentially incompressible and the conduit between the pressure sensor 216 and the reservoir 202 can be considered to be free of obstruction. In some implementations one or more of the pressure sensors 212, 214, 216 can be contained with the housing 210.

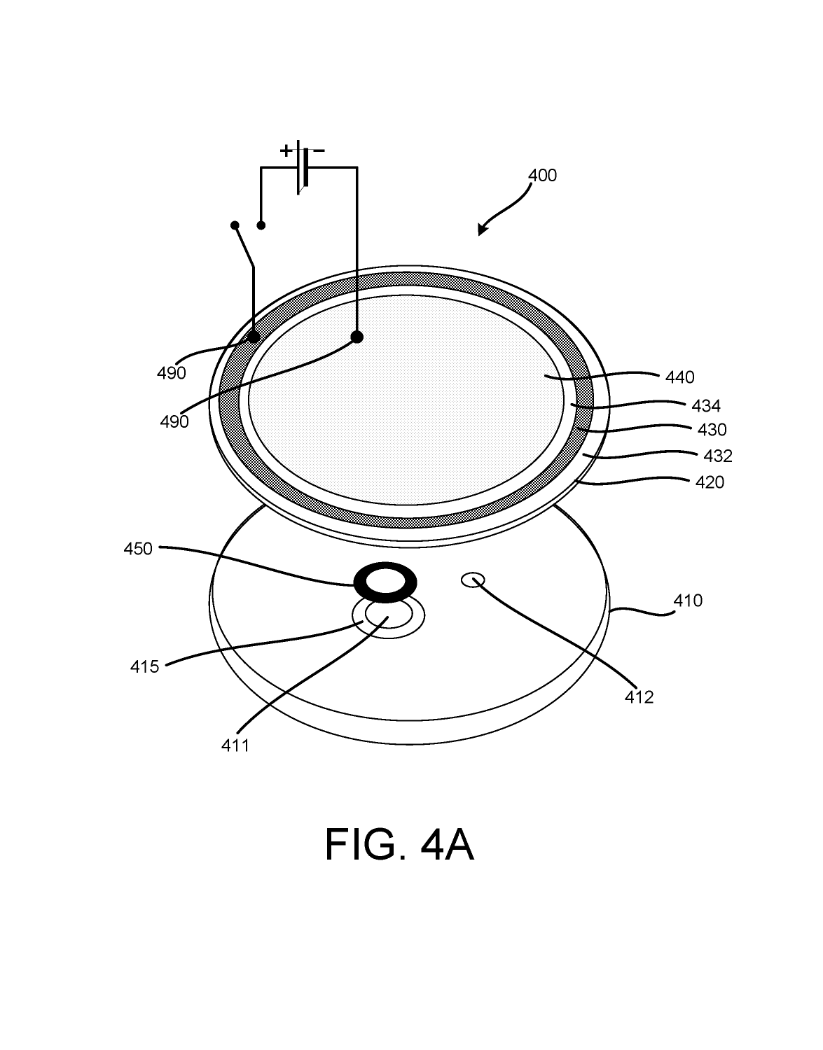

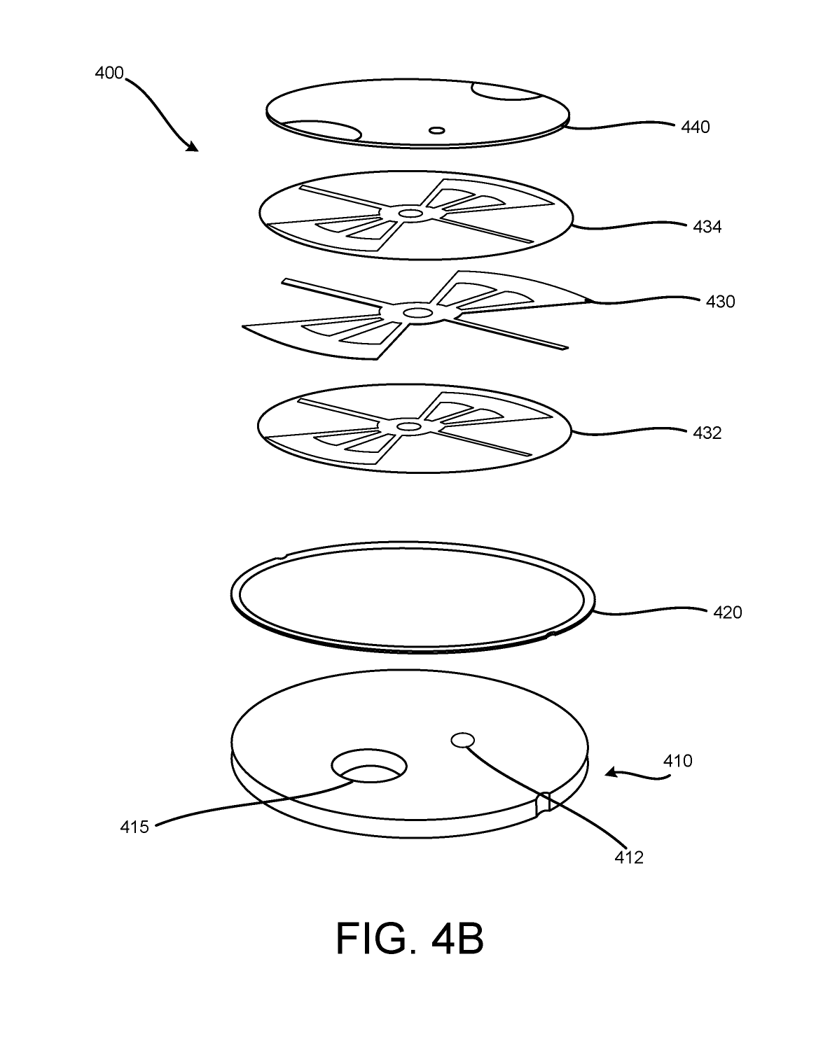

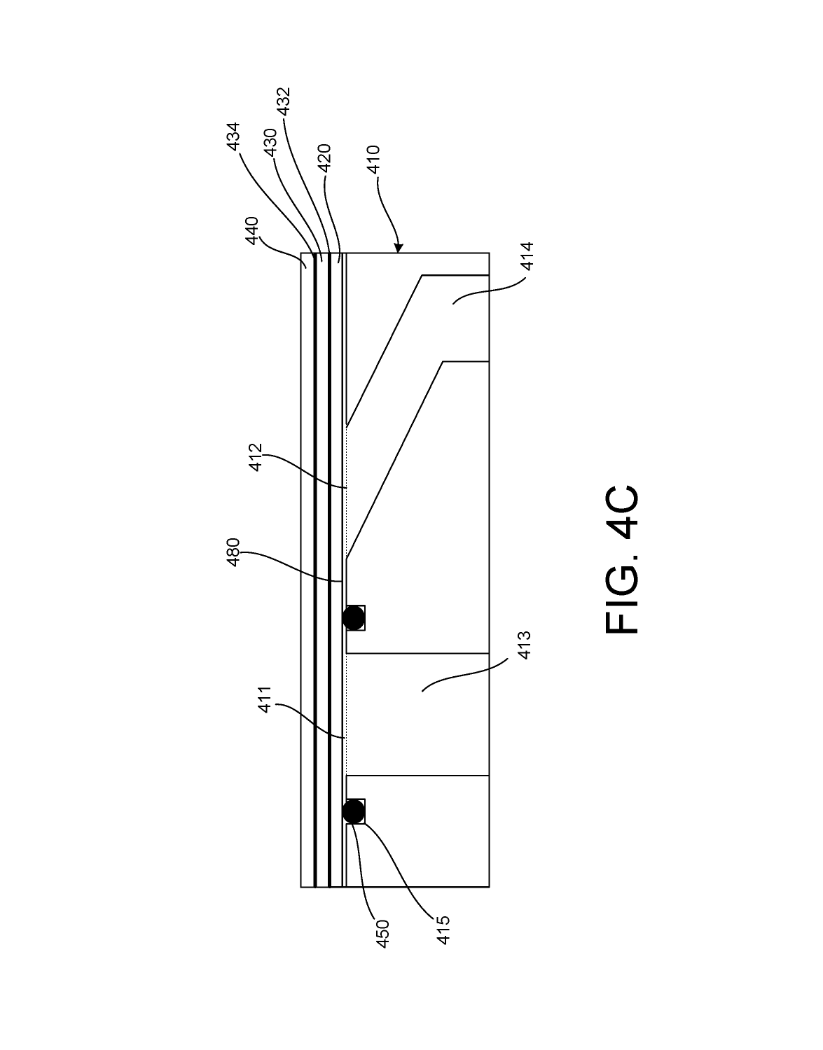

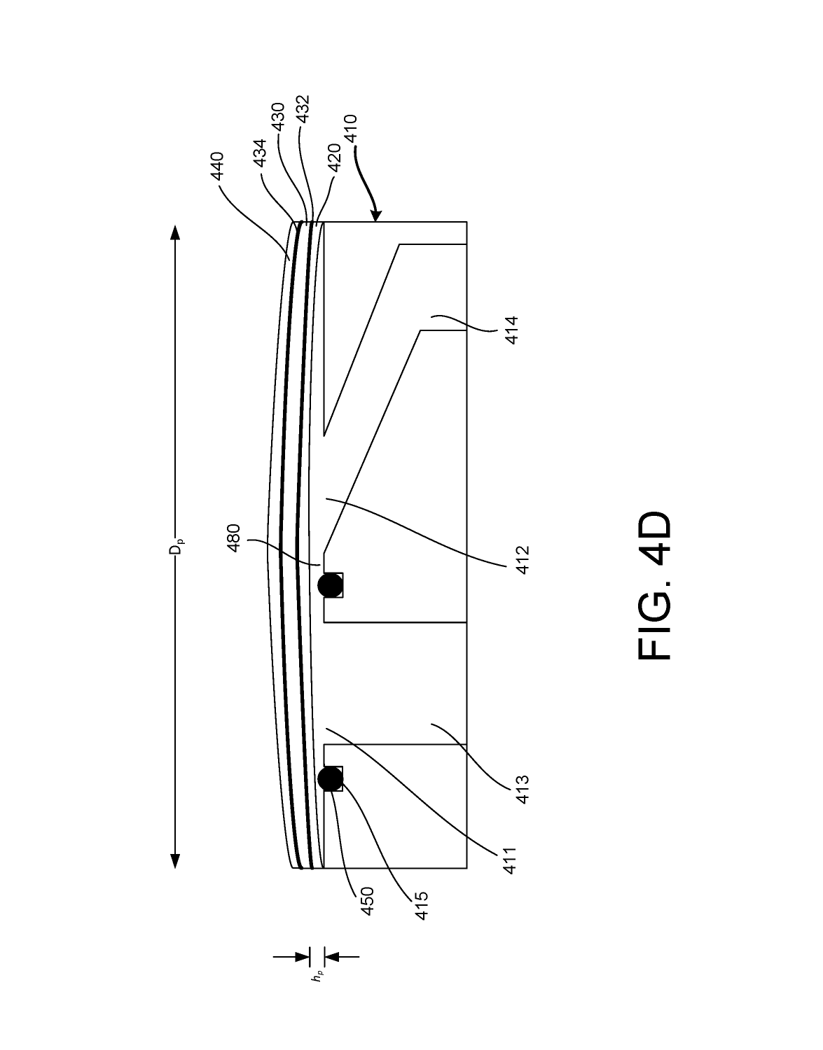

FIG. 4A is a partially exploded perspective view of an example valve device 400. FIG. 4B is an exploded perspective view of the example valve device 400. FIGS. 4C and 4D are cross-sectional views of the example valve device 400 shown in FIG. 4A, in an assembled state. The example valve device 400 shown in FIGS. 4A-4D is an example of a fluid control device, or a fluidic component, included in the fluid control system 206 of the example electronically controlled fluid manifold 230 described above.

In the example arrangement shown in FIGS. 4A-4D, the example valve device 400 includes a base plate 410 defining a base portion of the valve device 400. A diaphragm 420 is positioned on the base plate 410. A piezoelectric element 440 is positioned on the diaphragm 420, with an isolation layer 430 positioned between the diaphragm 420 and the piezoelectric element 440. The piezoelectric element can be electrically powered (e.g., by a battery in the implantable fluid-operated inflatable device 100) to drive the diaphragm 420 to open and close the valve device 400. The diaphragm 420 can include a thin metal foil, whose shape can be repeatably deformed in response to movement by the piezoelectric element 440. In some implementations, the diaphragm 420 can include titanium material. In some implementations, the diaphragm 420 can include gold material. In some implementations, the diaphragm 420 can include stainless steel material or other alloys. In some implementations, the isolation layer 430 can include a polyamide material that has a high resistivity, for example, a resistivity greater than 10¹³ Ohm-cm to provide electrical isolation between the piezoelectric element 440 and the diaphragm 420.

In some examples, an epoxy layer 432 provides for the coupling of the isolation layer 430 and the diaphragm 420. In some examples, an epoxy layer 434 provides for the coupling of the piezoelectric element 440 and the isolation layer 430, and the epoxy layers 432, 434 together provide for the coupling of the piezoelectric element 440 to the diaphragm 420. In some implementations, the epoxy layers 432, 434 are not distinct but are part of one epoxy layer. The epoxy layers 432, 434 can be formed from a mixture of different chemicals (e.g., a resin and a hardener) that, when mixed and cured, react to form a covalent bond and that adhere to surfaces that they contact. Curing of the epoxy can be controlled through selection of the resin and hardener chemicals used in the mixture, selection of the ratio of the chemicals used in the mixture, control of the temperature of the mixture, and application of electromagnetic radiation to the mixture.

In some examples, one or more electrodes 490 are arranged on the example valve device 400. In the example shown in FIG. 4A, the example valve device 400 includes a pair of electrodes 490 coupled between the isolation layer 430 and the piezoelectric element 440. Application of a voltage to the piezoelectric element 440 causes a deflection or deformation of the piezoelectric element 440 and a corresponding deflection or deformation of the diaphragm 420 coupled thereto.

In the example arrangement shown in FIGS. 4A-4D, a fluid chamber 480 is defined between the base plate 410 and the diaphragm 420. For example, in some implementations, the diaphragm 420 can be bonded to the base plate 410 at the periphery of the diaphragm to form a fluid-tight connection between the base plate 410 and the diaphragm 420. The base plate 410 includes a first opening 411 that provides for communication between a first fluid passageway 413 and the fluid chamber 480. The base plate 410 includes a second opening 412 that provides for communication between a second fluid passageway 414 and the fluid chamber 480. In the example arrangement shown in FIGS. 4A-4D, the base plate 410 includes a recess 415 surrounding the first opening 411, with a seal 450, in the form of an O-ring in the example shown in FIGS. 4A-4D, fitted in the recess 415. In some examples, a top portion of the seal 450 is pressed against the diaphragm 420 in the closed position of the valve device 400, as shown in FIG. 4C to close off the chamber 480 and inhibit the flow of fluid through the example valve device 400, between the first fluid passageway 413 and the second fluid passageway 414 via the chamber 480. In some examples, in which the valve device 400 does not include a seal 450, the diaphragm 420 is seated against the base plate 410 to close off the chamber 480 and inhibit the flow of fluid through the valve device 400. In the open position of the example valve device 400, the base plate 410 and the top portion of the seal 450 are separated, or spaced apart from, the diaphragm 420 due to the deflection of the diaphragm 420. This positioning of the seal 450 and the base plate 410 relative to the diaphragm 420 opens the chamber 480 and allows fluid to flow through the example valve device 400, between the first fluid passageway 413 and the second fluid passageway 414 via the fluid chamber 480. In the case of a circular diaphragm 420, the fluid chamber 480 can have a diameter, Dₚ, and a height, hₚ, that depends on the voltage of the piezoelectric element 440 that is actuated to change the shape of the diaphragm.

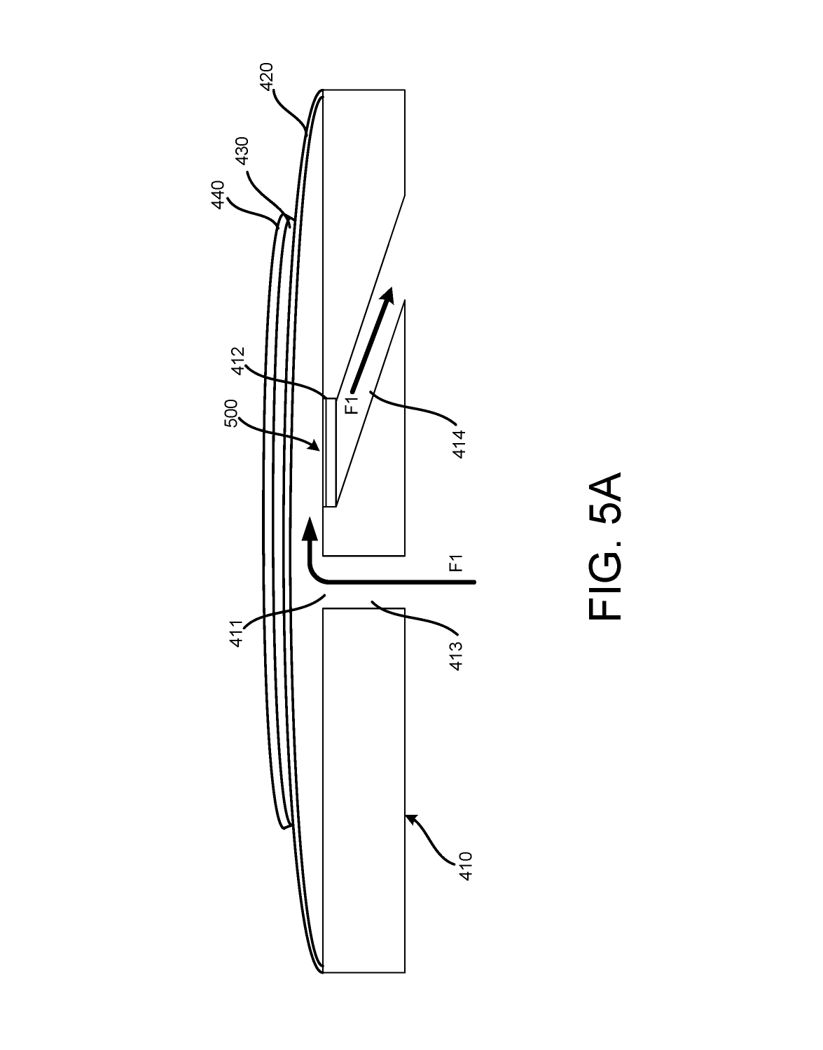

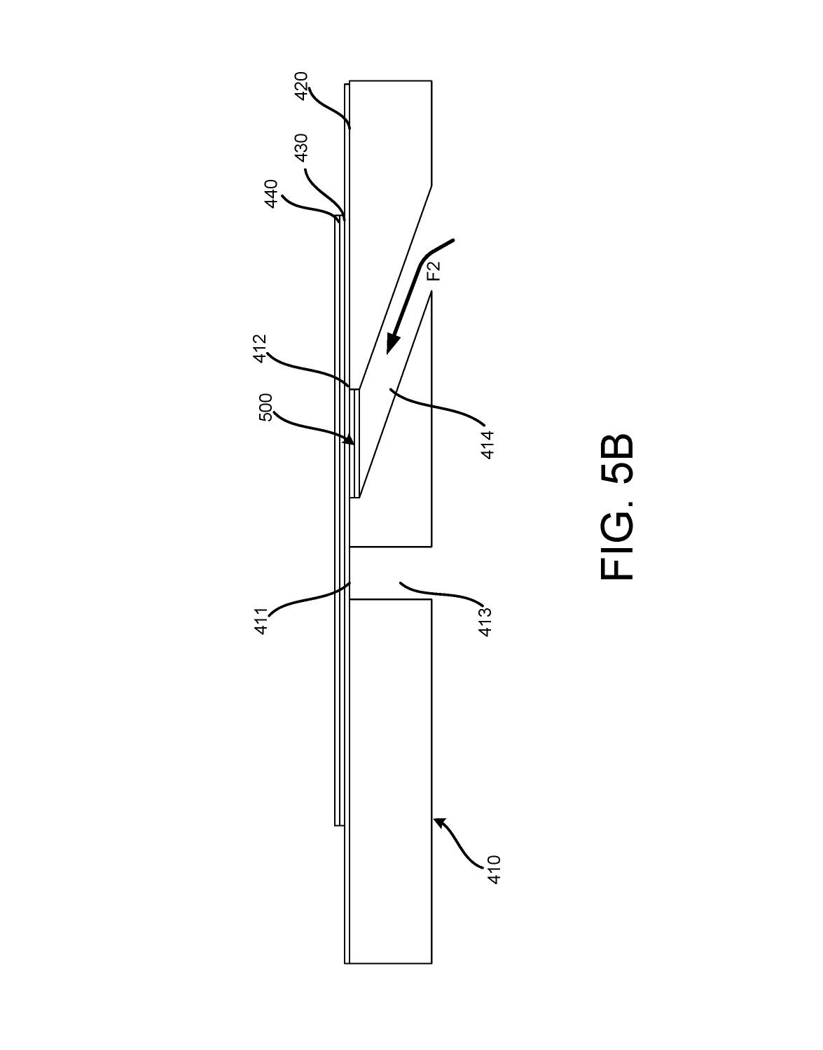

FIGS. 5A and 5B are cross-sectional views of the example valve device 400 shown in FIGS. 4A-4D, including an example flow control device 500 positioned in one of the fluid passageways of the example valve device 400.

FIG. 5A illustrates an example in which the valve device 400 is open, allowing fluid to flow in the direction of the arrows F1, through the first fluid passageway 413, into the chamber 480, and out of the valve device 400 through the second fluid passageway 414. The example shown in FIG. 5A may illustrate an open position of the valve device 400 that allows fluid to flow, for example, from the reservoir 202 to the inflatable member 204 to provide for inflation/pressurization of the inflatable member 204.

In the example arrangement shown in FIGS. 5A and 5B, the example flow control device 500 is positioned at the second opening 412 formed in the base plate 410, the second opening 412 providing for fluid communication between the fluid chamber 480 and the second fluid passageway 414. In some examples, the flow control device 500 is a check valve, or a one-way valve, which allows for flow in one direction (in this example, in the direction of the arrows F1), while inhibiting flow in the opposite direction.

FIG. 5B illustrates the closed position of the valve device 400, in which the flow of fluid through the valve device 400 is blocked. In some examples, the closed position shown in FIG. 5B may maintain an inflation pressure of the inflatable member 204. As described above, in some situations, pressure fluctuations and/or pressure spikes may exert a force, or pressure on the valve device 400 in the closed position. FIG. 5B illustrates a pressure spike, or a back pressure, exerted in the direction of the arrow F2. In the example described above with respect to FIGS. 4A-4D, this type of pressure spike, or back pressure exerted on the diaphragm 420/piezoelectric element 440 could cause an unintentional opening of the valve device 400, and an unintentional deflation/depressurization of the inflatable member 204. In the example shown in FIG. 5B, the flow control device 500 (positioned at the second opening 412, between the second fluid passageway 414 and the fluid chamber 480), for example, in the form of a check valve or a one-way valve, remains in the closed position in response to the pressure spike/back pressure/flow of fluid in the direction of the arrow F2. Thus, the positioning of the flow control device 500 at the second opening 412, allowing flow in a first direction, i.e., the direction of the arrows F1, while blocking flow in a second direction, i.e., the direction of the arrow F2, maintains the closed state of the valve device 400, even in response to fluctuation in pressure, or pressure spike, or back pressure.

The general architecture and principles of operation of the valve device described above also can be used to implement one or more pumps (such as pumps P1, P2 of FIG. 3) to pump fluid from one location to another. For example, repeated movement of a diaphragm between an open position and a closed position, relative to a base plate, can cause fluid to be drawn into a chamber formed between the diaphragm and the base plate through a first fluid passageway and expelled out of the chamber into a second fluid passageway. In this manner, fluid can be pumped from a first location that is fluidically connected to the first passageway to a second location that is fluidically connected to the second passageway. In some implementations, one or more one-way valves can be configured to prevent, or limit, the flow of fluid in the direction from the second location to the first location.

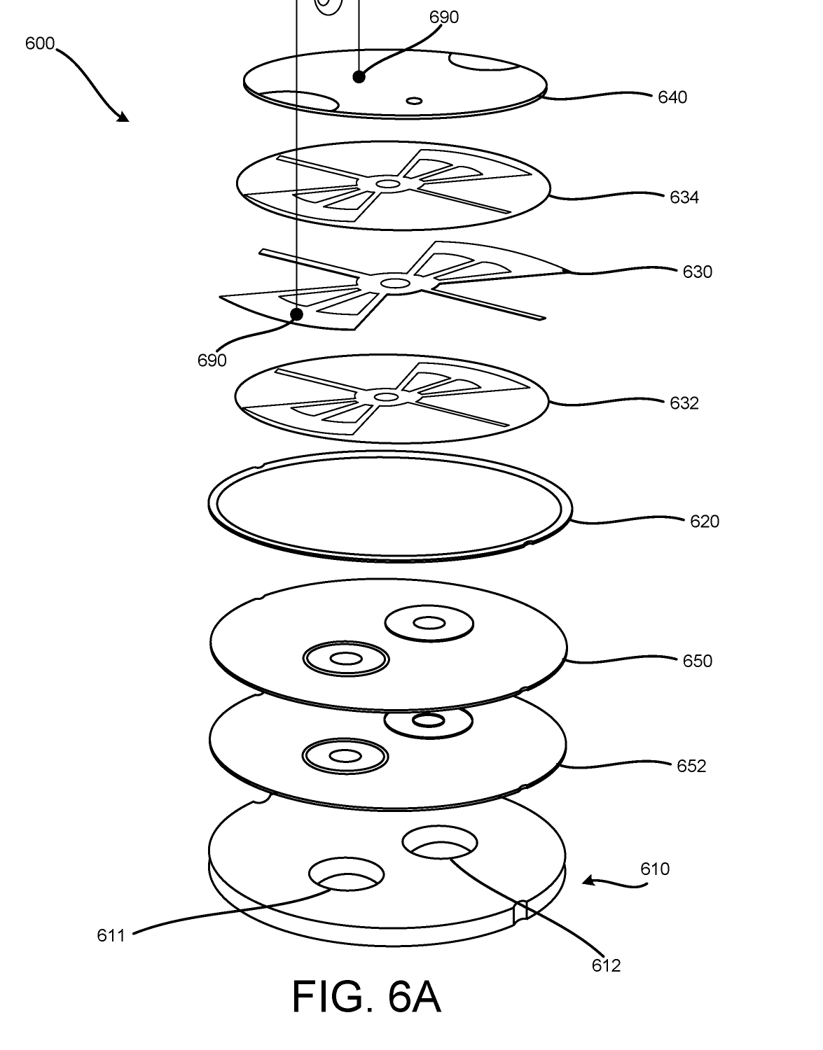

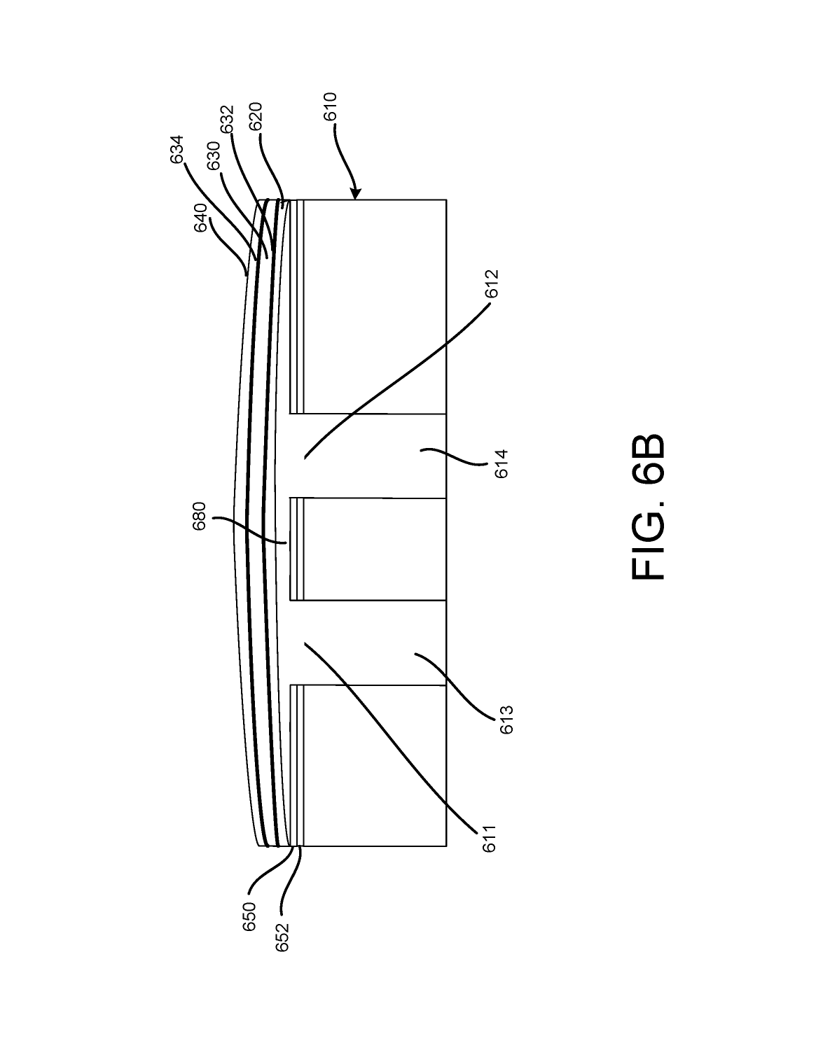

FIG. 6A is a partially exploded perspective view of an example pump device 600, and FIG. 6B is a cross-sectional view of the example pump device 600. The example pump device 600 shown in FIGS. 6A-6B is an example of a fluid control device, or a fluidic component, included in the fluid control system 206 of the example electronically controlled fluid manifold 230 described above.

In the example arrangement shown in FIGS. 6A-6B, the example pump device 600 includes a base plate 610 defining a base portion of the pump device 600. A diaphragm 620 is positioned on the base plate 610. A piezoelectric element 640 is positioned on the diaphragm 620, with an isolation layer 630 positioned between the diaphragm 620 and the piezoelectric element 640. The piezoelectric element can be electrically powered (e.g., by a battery of the implantable fluid-operated inflatable device 100) to drive the diaphragm 620 to pump fluid through the pump device 600. The diaphragm 620 can include a thin metal foil, whose shape can be repeatably deformed in response to movement by the piezoelectric element 640. In some implementations, the diaphragm 620 can include titanium material. In some implementations, the diaphragm 620 can include gold material. In some implementations, the diaphragm 620 can include stainless steel material or other alloys. In some implementations, the isolation layer 630 can include a polyamide material that has a high resistivity, for example, a resistivity greater than 10¹³ Ohm-cm to provide electrical isolation between the piezoelectric element 640 and the diaphragm 620.

In some examples, an epoxy layer 632 provides for the coupling of the isolation layer 630 and the diaphragm 620. In some examples, an epoxy layer 634 provides for the coupling of the piezoelectric element 640 and the isolation layer 630, and the epoxy layers 632, 634 together provide for the coupling of the piezoelectric element 640 to the diaphragm 620. In some implementations, the epoxy layers 632, 634 are not distinct but are part of one epoxy layer. The epoxy layers 632, 634 can be formed from a mixture of different chemicals (e.g., a resin and a hardener) that, when mixed and cured, react to form a covalent bond and that adhere to surfaces that they contact. Curing of the epoxy can be controlled through selection of the resin and hardener chemicals used in the mixture, selection of the ratio of the chemicals used in the mixture, control of the temperature of the mixture, and application of electromagnetic radiation to the mixture.

In some examples, one or more electrodes 690 are arranged on the example pump device 600. In the example shown in FIG. 6A, the example pump device 600 includes a pair of electrodes 690 coupled between the isolation layer 630 and the piezoelectric element 640. Application of a voltage to the piezoelectric element 640 causes a deflection or deformation of the piezoelectric element 640 and a corresponding deflection or deformation of the diaphragm 620 coupled thereto.

When the pump device 600 is used in the fluid control system 206 of the example electronically controlled fluid manifold 230 described above, the piezoelectric element 640 can be controlled to cause fluid to be pumped by device 600, for example, by repeatedly changing a volume of the fluid chamber 680 by deforming the deformable diaphragm 620 to pump fluid from the fluid reservoir to the inflatable member.

In the example arrangement shown in FIGS. 6A-6B, a fluid chamber 680 is defined between the base plate 610 and the diaphragm 620. The base plate 610 includes a first opening 611 that provides for communication between a first fluid passageway 613 and the fluid chamber 680. The base plate 610 includes a second opening 612 that provides for communication between a second fluid passageway 614 and the fluid chamber 680. In some examples, the diaphragm 620 can be actuated to move between a closed position in which the diaphragm 620 is proximate to the base plate 610 due to the deflection of the diaphragm 620, such that the volume of the chamber 680 is minimized, and an open position in which the base plate 610 is separated, or spaced apart from, the diaphragm 620 due to the deflection of the diaphragm 620, such that the volume of the chamber is maximized. When the diaphragm 620 is actuated to move from the closed position to the open position, fluid can be drawn into the chamber 680 through the first fluid passageway 613, and when the diaphragm 620 is actuated to move from the open position to the closed position, fluid can be expelled from the chamber 680 through the second fluid passageway 614. Repeatedly actuating the diaphragm between the closed and open position allows fluid to be pumped through the pump device 600, from the first fluid passageway 613 to the second fluid passageway 614 via the fluid chamber 680.

In some implementations, the pump device 600 can include one or more foil plates 650 and 652 to control the flow of fluid into and out of the pump device 600. The foil plates 650, 652 can include one-way check valves that operate to permit fluid to flow in one direction through the valves but not in an opposite direction. The one-way check valves defined by the one or more foil plates can be positioned in, or in fluid connection with, a fluid passageway 613, 614 of the pump device 600. In some examples, a check valve is positioned in, or in fluid connection with, a portion of a fluid passageway 613, 614 so as to inhibit the unintended flow of fluid through the pump device in the event of a fluctuation, or spike in pressure. In some examples, a check valve is positioned in a fluid passageway 613, 614 so as to counteract a back pressure that would otherwise overcome the closing pressure and cause unintentional flow through the pump device 600. In some example implementations, a first check valve defined by one or more foil plates 650, 652 is positioned in, or in fluid connection with (e.g., at a first opening 611 of), a first fluid passageway 613 of the pump device and is configured to permit fluid to easily flow from the first fluid passageway 613 into the chamber 680 but to prevent or inhibit the flow of fluid from the chamber 680 into the passageway 613. In some example implementations, a second check valve defined by one or more foil plates 650, 652 is positioned in, or in fluid connection with (e.g., at a first opening 612 of), a second fluid passageway 614 of the pump device 600 and is configured to permit fluid to easily flow from the chamber 680 into the second fluid passageway 614 but to prevent or inhibit the flow of fluid from the passageway 614 into the chamber 680.

Application of an alternating current (AC) voltage to the piezoelectric element 640 can cause the diaphragm 620 of the pump device 600 to oscillate between a first position that defines the closed position of the chamber 680, in which the diaphragm 620 is proximate to the base plate 610 and the volume of the chamber 680 is minimized, and a second (e.g., domed) position that defines the open position of the chamber 680, in which the diaphragm 620 is separated from the base plate and the volume of the chamber 680 is maximized. As the diaphragm 620 of the pump device 600 oscillates between a first position and the second position, fluid is drawn into the chamber 680 from the first passageway 613 and is expelled from the chamber 680 into the second passageway 614. As the diaphragm 620 of the pump device 600 oscillates between a first position and the second position, the one-way check valves defined by the one or more foil plates 650, 652 prevent or inhibit fluid from flowing from the chamber 680 into the first passageway 613 and prevent or inhibit fluid from flowing into the chamber 680 from the second passageway 614. Thus, the application of the AC voltage to the piezoelectric element 640 causes the pump device 600 to pump fluid from the first passageway 613 to the second passageway 614.

The frequency of the AC voltage applied to the piezoelectric element 640 can determine an oscillation mode of the piezoelectric element 640. In some implementations, the frequency of the AC voltage is selected to excite a lowest-order mode in which the center of the circular piezoelectric element 640 experiences the greatest extent of movement during an oscillation cycle, such that an amount of fluid pumped during an oscillation cycle is maximized compared to other oscillation modes.

The piezoelectric element 640 can be controlled to cause fluid to be pumped by device 600, for example, by repeatedly changing a volume of the fluid chamber 680 by deforming the deformable diaphragm 620 to pump fluid from the fluid reservoir to the inflatable member.

The volume of the chamber 680 can be determined, at least in part, by the shape, geometry, and material properties of the components used to form the chamber 680, including, for example, the base plate 610 and the deformable diaphragm 620. In some cases, a relatively larger volume of the chamber 680, for an approximately constant diameter of the chamber, can result in more fluid being pumped in each open/close cycle of the pump device 600. To achieve a relatively larger volume of chamber 680, the deformable diaphragm can be deformed or biased into a non-flat dome-shaped configuration before it is attached to the piezoelectric element 640.

In some implementations, before the diaphragm 620 is attached to the piezoelectric element 640, a voltage can be placed across the electrodes 690 attached to the piezoelectric element 640 to configure the piezoelectric element 640 in the domed configuration that is assumed when the fluid chamber is in the open position (See FIG. 4D). Then, the diaphragm can be placed in contact with the piezoelectric element while the piezoelectric element 640 is in its domed configuration, and the epoxy can be cured when the piezoelectric element and the diaphragm 620 are in the domed configuration, which can reduce stress on the adhesive bond between the diaphragm 620 and the piezoelectric element 640.

Referring again to FIGS. 2A and 2B, although considerable effort is expended to maintain the cleanliness of the components of the system and the purity of the fluid used within the system, it is still possible that some small amounts of foreign matter can contaminate the fluid within the system. For example, when the reservoir 202, the inflatable members 204, and the housing 210 are implanted and connected (e.g., by conduits 203, 207) within a patient, it is possible that some contamination enters the fluidic system. In addition, it is possible that, once implanted within a patient, that small amounts of material disintegrate from walls of the reservoir 202, inflatable member 204, housing 210 and conduits 203, 207 and become suspended within fluid that flows within the implantable fluid-operated inflatable device 200. Because of the small internal dimensions of the pumps and valves used within the fluidic system, the existence of particles of foreign matter suspended within the fluid flowing within the system poses a risk of clogging or damaging one or more of the pumps and valves, which may lead to malfunction of the implantable fluid-operated inflatable device 200. To mitigate the effect of any particulate matter suspended within the fluid that flows within the implantable fluid-operated inflatable device 200, the fluidic path can include one or more filters that block, or reduce the amount of, particulate matter that enters the pumps and valves of the system. In some implementations, the filters can be included in a fluid pathway of a pump or valve.

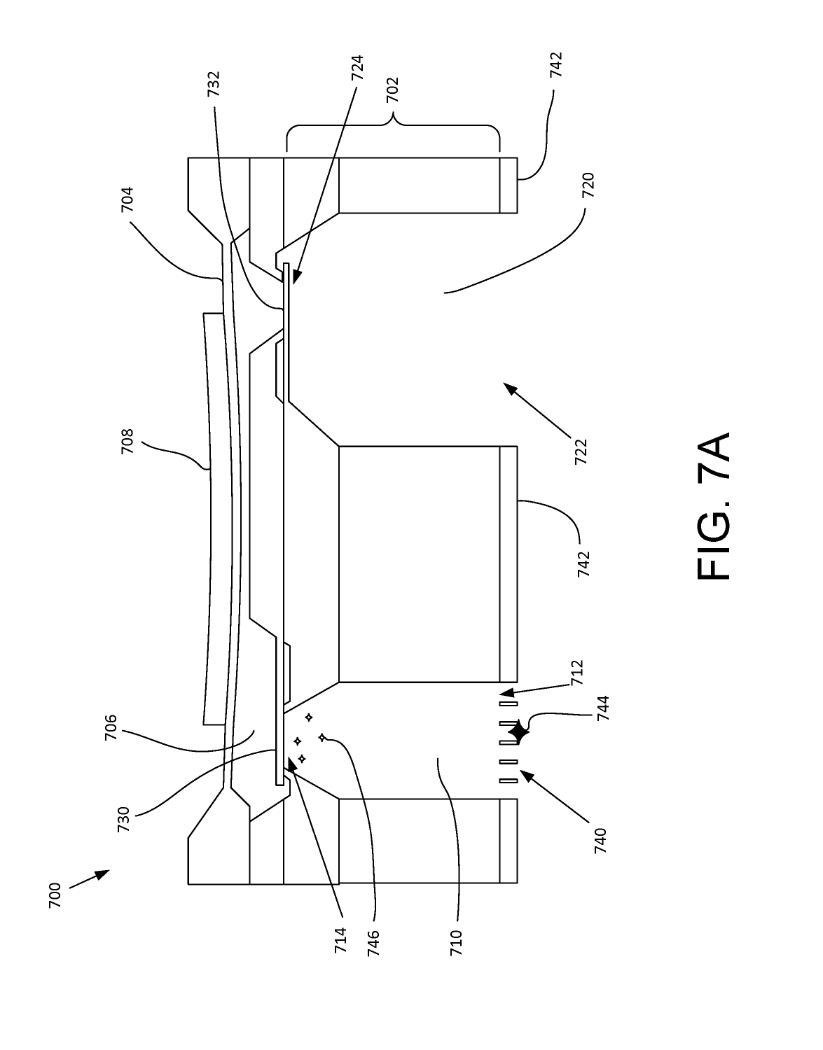

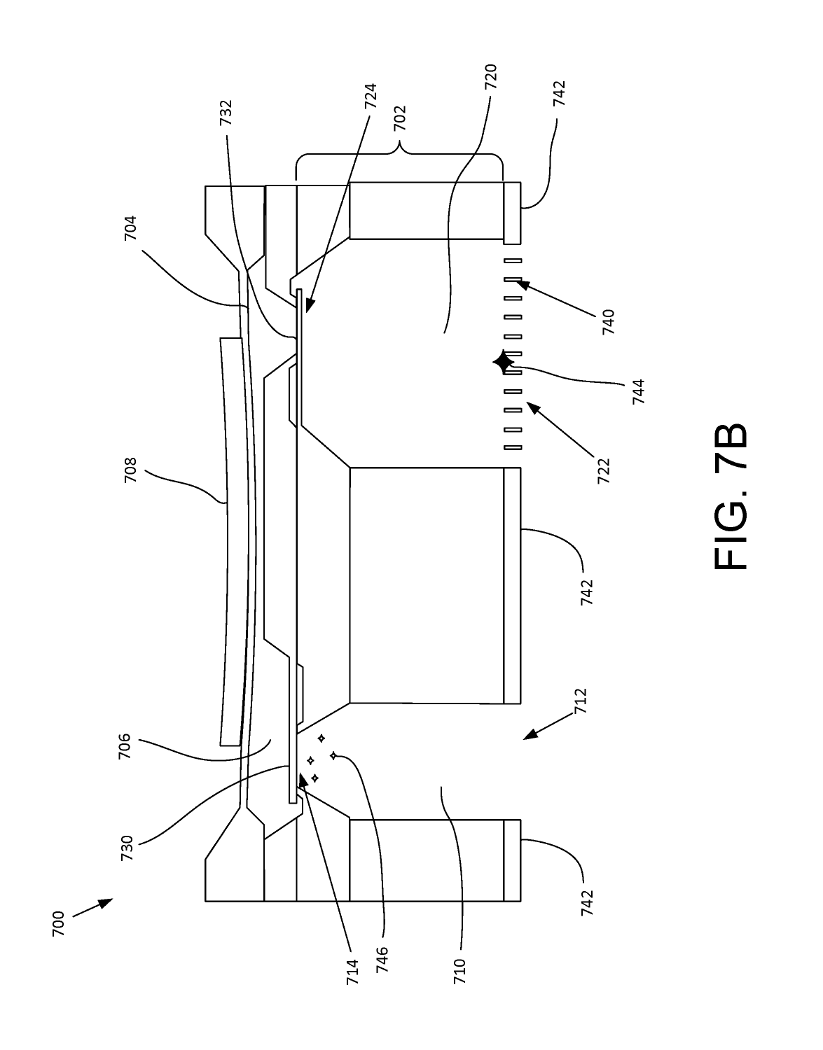

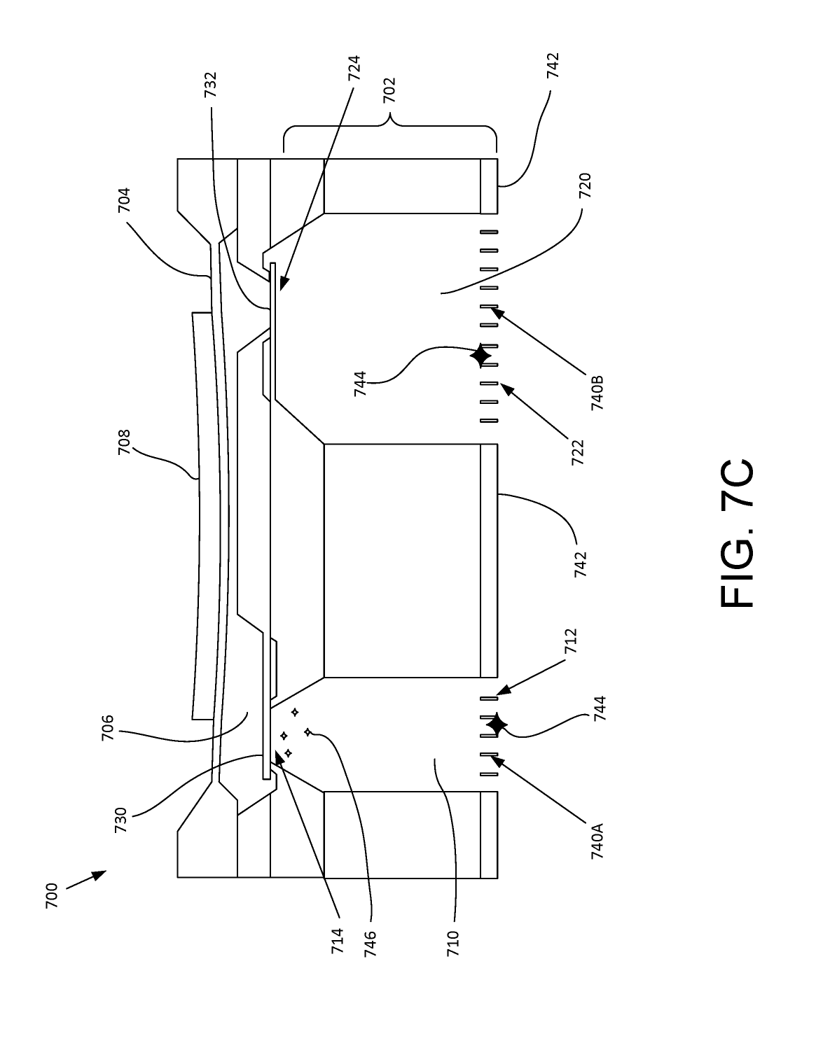

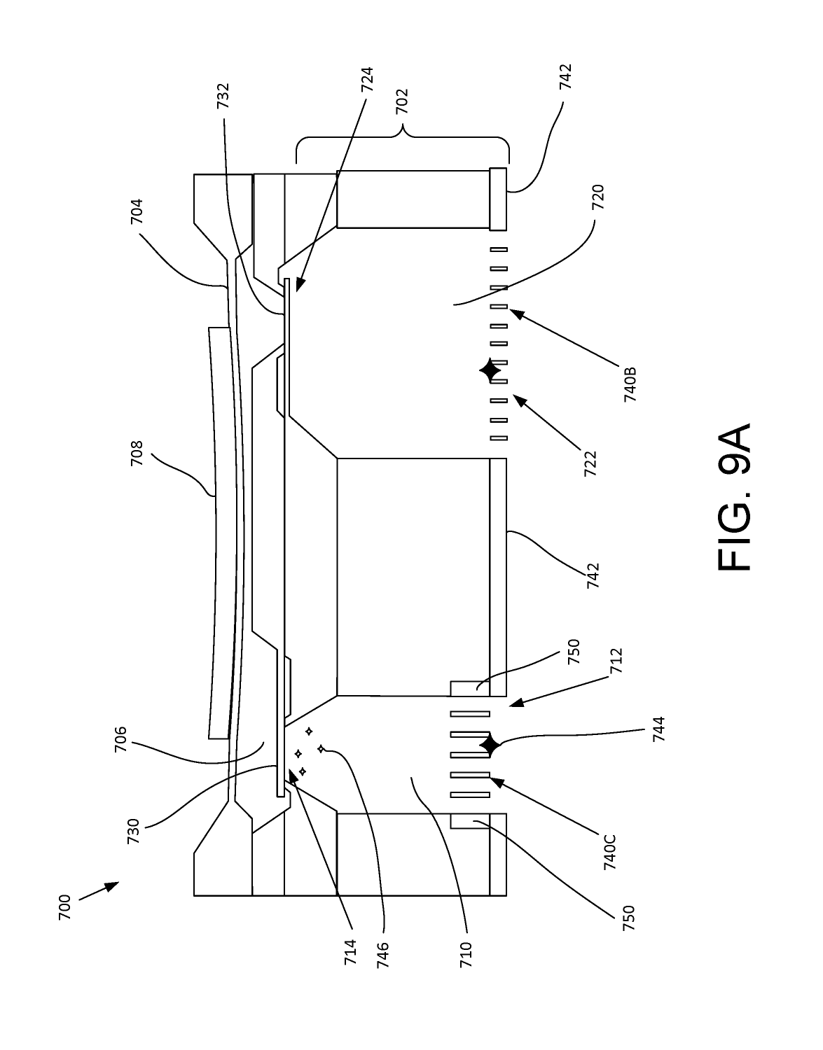

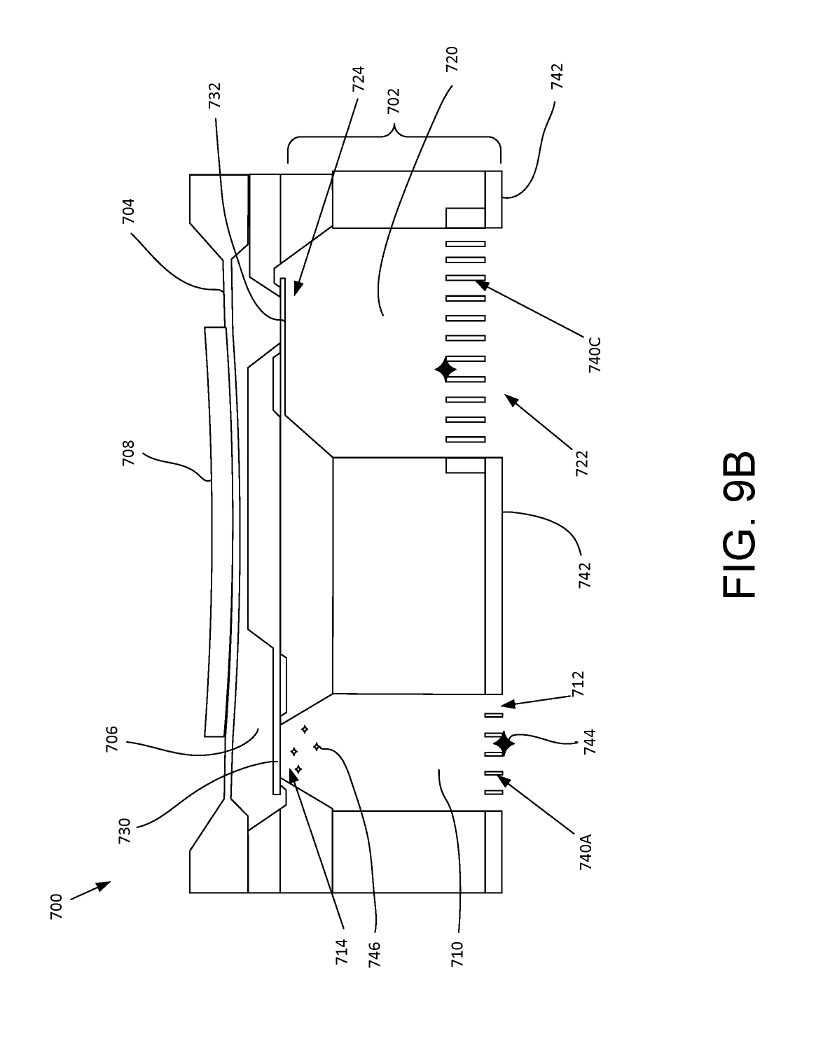

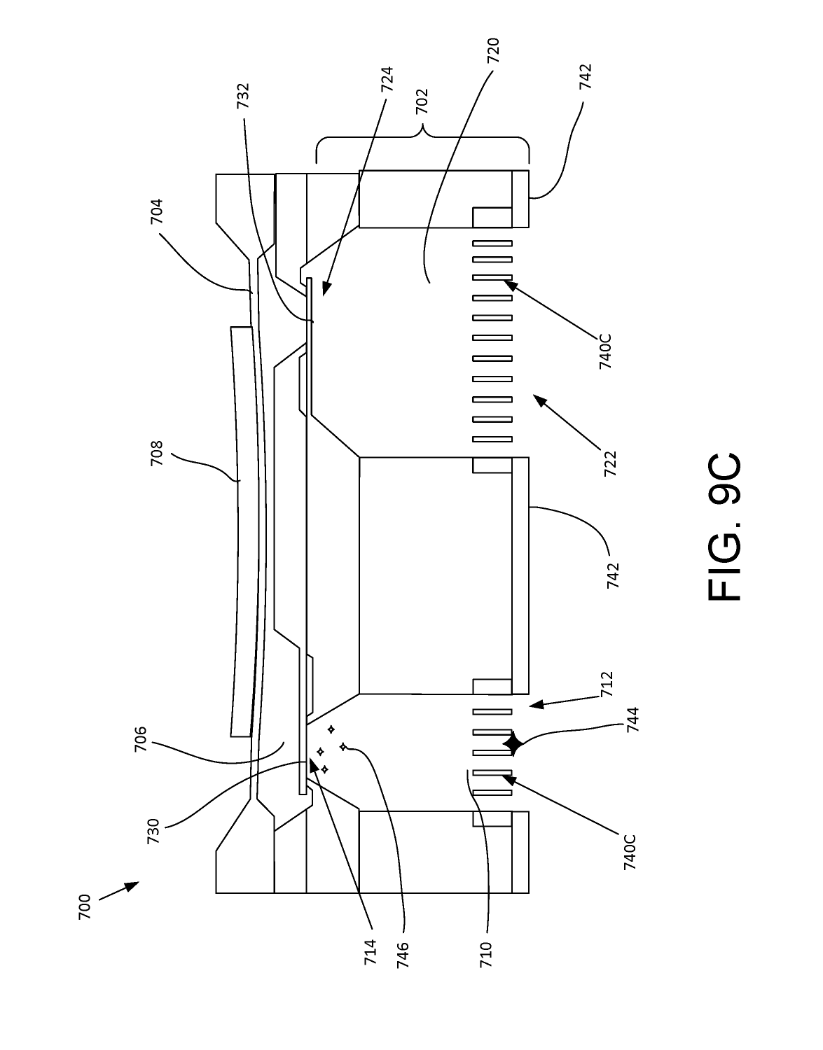

FIGS. 7A, 7B, 7C, 9A, 9B, and 9C are cross-sectional views of example pump devices 700 that includes a filter for capturing particulate matter in the fluid flow and/or for blocking the particulate matter from entering certain parts of the fluidic system (e.g., for blocking particulate matter from entering a pump chamber of the device). The example pump device 700 shown in FIGS. 7A, 7B, 7C, 9A, 9B, and 9C are examples of a fluid control device, or a fluidic component, included in the fluid control system 206 of the example electronically controlled fluid manifold 230 described above.

In the example arrangements shown in FIGS. 7A, 7B, 7C, 9A, 9B, and 9C, the example pump device 700 includes a base plate 702 defining a base portion of the pump device 700. A diaphragm 704 is positioned above the base plate 702, and a fluid chamber 706 is defined between the base plate 702 and the diaphragm 704. A piezoelectric element 708 is positioned on the diaphragm 704. The piezoelectric element can be electrically powered (e.g., by a battery of the implantable fluid-operated inflatable device) to drive the diaphragm 704 to pump fluid through the pump device 700. The diaphragm 704 can include a thin metal foil, whose shape can be repeatably deformed in response to movement by the piezoelectric element 708. In some implementations, the diaphragm 704 can include titanium material.

The base plate 702 can define a first fluid passageway 710 through which fluid can flow from a fluid reservoir into the fluid chamber 706. The first fluid passageway 710 can include an opening 712 at a first end of the passageway 710, which is distal to the fluid chamber 706, and can include an opening 714 and a second end of the passageway 710, which is proximate to the fluid chamber 706. The base plate 702 can define a second fluid passageway 720 through which fluid can flow from the fluid chamber 706 to an inflatable member. The second fluid passageway 720 can include an opening 722 at a first end of the passageway 720, which is distal to the fluid chamber 706, and can include an opening 724 and a second end of the passageway 720, which is proximate to the fluid chamber 706. In some implementations, the first fluid passageway 710 and the second fluid passageway 720 can be tapered, such the passageways 710, 720 have larger cross-sectional areas at the ends 712, 722 of the passageways that are distal to the fluid chamber 706 than at ends of the passageways that are proximate to the fluid chamber.

The pump device 700 can include a first flexible flap 730 that includes a portion that has an area that is greater than an area of the passageway opening 714 that is proximate to the fluid chamber 706 and that covers the opening, such that the first flexible flap 730 is configured to seal against portions of the base plate that defines the opening 714 of the first fluid passageway 710 to close the opening 714 when a fluid pressure in the fluid chamber 706 is greater than a fluid pressure of fluid in the first fluid passageway 710. The flexible flap 730 can be secured to the base plate over a portion of its extent but can have a portion that is unsecured, such that at least a portion of the flexible flap is configured to be pushed away from one or more walls of the fluid passageway 710 that defines the opening 714 when a fluid pressure of fluid in the first fluid passageway 710 is greater than a fluid pressure in the fluid chamber 706. In this manner, the flexible flap 730 operates to allow fluid to flow from the first fluid passageway 710 into the fluid chamber 706 but to block the flow of fluid from the fluid chamber 706 into the first fluid passageway 710. The flexible flap 730 can be made of a variety of materials including, for example, titanium, elastomeric material, plastic material, etc. The pump device 700 can include a second flexible flap 732 that includes a portion that has an area that is greater than an area of the passageway opening 724 that is proximate to the fluid chamber 706 and that covers the opening, such that the second flexible flap 732 is configured to seal against portions of the base plate that defines the opening 724 of the second fluid passageway 720 to close the opening 724 when a fluid pressure in the fluid chamber 706 is less than a fluid pressure of fluid in the second fluid passageway 720. The flexible flap 732 can be secured to the base plate over a portion of its extent but can have a portion that is unsecured, such that at least a portion of the flexible flap is configured to be pushed away from one or more walls of the second fluid passageway 720 that defines the opening 724 when a fluid pressure in the fluid chamber 706 is greater than a fluid pressure of fluid in the second fluid passageway 720. In this manner, the flexible flap 732 operates to allow fluid to flow from the fluid chamber 706 into the second fluid passageway 720 but to block the flow of fluid from the second fluid passageway 720 into the fluid chamber 706. The flexible flap 732 can be made of a variety of materials including, for example, titanium, elastomeric material, plastic material, etc.

With the flexible flaps 730, 732 configured in this way to allow fluid to flow in a first direction from the first fluid passageway 710 into the fluid chamber 706 and out of the fluid chamber into the second fluid passageway 720 but not in a direction opposite to the first direction, repeated expansion and contraction of the volume of the fluid chamber 706 in response to the piezoelectric element 708 operating on the deformable diaphragm 704 can cause fluid to be pumped from a reservoir fluidically connected to the first fluid passageway 710 to an inflatable member that is fluidically connected to the second fluid passageway 720.

The pump device 700 can include a fluid filter 740 that is located within, or at the end 712 of, the first fluid passageway 710 or that is located within, or at the end 722 of, the second fluid passageway 720. The fluid filter 740 can operate to block, for example, debris, foreign matter, particulates suspended in the fluid flowing through the device 700 from passing through the first fluid passageway 710 and into the fluid chamber 706 and/or from exiting the second fluid passageway 720. For example, as shown in FIG. 7A, a fluid filter 740 is located at the opening 712 into the first fluid passageway 710. As shown in FIG. 7B, a fluid filter 740 is located at the opening 722 into the second fluid passageway 720. As shown in FIG. 7C, a first fluid filter 740A is located at the opening 712 into the first fluid passageway 710, and a second fluid filter 740B is located at the opening 722 into the second fluid passageway 720.

In some implementations, the fluid filters 740, 740A, 740B can include a metal foil (e.g., a titanium foil, having a pattern of openings that permit fluid to flow through the openings but that block particulates having a characteristic size larger than a threshold size from flowing through the opening. For example, particulates 744 having a characteristic size (e.g., minimum transverse extent) that is greater than a threshold size defined by the size (e.g., diameter) of the openings can be blocked by the filters 740, 740A, 740B, while particulates 746 and a characteristic size smaller than the threshold size can pass through the filters 740, 740A, 740B.

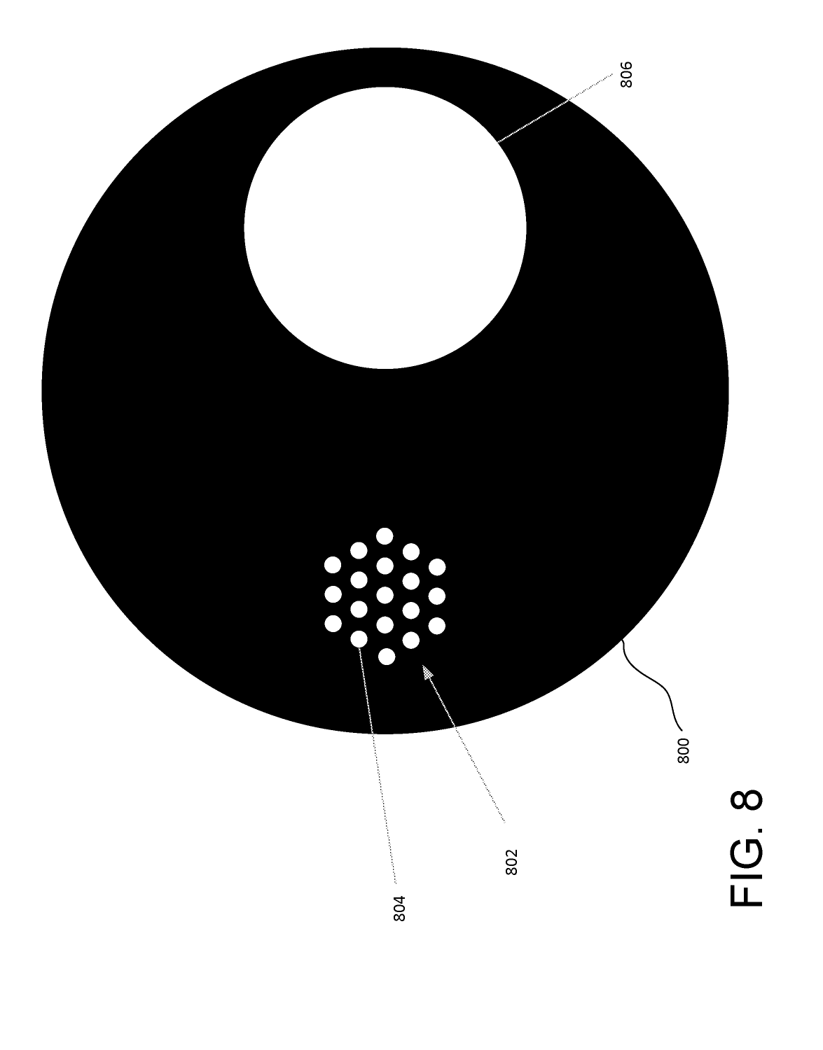

FIG. 8 is a schematic end view of a filter foil 800. In some implementations, the filter foil 800 can be made of metal (e.g., titanium) and can have a first section 802 that includes a plurality of openings 804. The openings can have a variety of different shapes, including circular, oblong, square, rectangular, hexagonal, etc. The plurality of openings 804 can be arranged in a regular or irregular pattern. For example, the openings 804 can be arranged in a two-dimensional hexagonal pattern, as shown in FIG. 8, or in a square pattern, or another type of regular or irregular pattern.

The plurality of openings 804 can be formed in the filter foil 800 in a number of different ways. For example, in some implementations, the pattern of openings can be mechanically stamped into the metal foil 800. In some implementations, the pattern of openings 804 can be laser etched into the metal foil 800. In some implementations, the pattern of openings can be chemically etched (e.g., through a lithographic process) into the metal foil 800.

Referring again to FIG. 7A and also to FIG. 8, the section 802 that includes the plurality of openings 804 can be arranged on the filter foil 800 so that the pattern of openings 804 is aligned with the opening 712 of the first fluid passageway 710 when the filter foil 800 is attached to the base plate 702. The filter foil 800 also can include an opening 806 in the filter foil that is aligned with the opening 722 of the second fluid passageway 720 of the base plate 702 when the filter foil is attached to the base plate.

In some implementations, the filter foil 800 can be welded to the base plate 702. For example, when the base plate includes titanium and the filter foil 800 includes titanium, the filter foil 800 can be welded to the titanium base plate 702. Prior to attempting (e.g., welding) the filter foil 800 to the base plate 702, the filter foil 800 can be positioned relative to the openings 712, 720 in the base plate, such that the first section 802 of the filter foil, which includes the plurality of openings 804, is positioned at the end of the first fluid passageway 710 and such that the opening 806 in the filter foil 800 is positioned at the end of the second fluid passageway 720. Similarly, when a filter foil is attached to the base plate shown in FIG. 7B, a section of the filter foil having a plurality of openings can be aligned with the end of the second fluid passageway 720, and a larger opening in the filter foil 800 can be aligned with the end of the first fluid passageway 710. Similarly, when a filter foil is attached to the base plate shown in FIG. 7C, a first section having a plurality of openings can be aligned with the end of the second fluid passageway 720 and a second section having a plurality of openings can be aligned with the end of the first fluid passageway 710.

In implementations in which the first fluid passageway 710 and the second fluid passageway 720 are tapered, such the passageways 710, 720 have larger cross-sectional areas at the ends 712, 722 of the passageways that are distal to the fluid chamber 706 than at ends of the passageways that are proximate to the fluid chamber, filters 740, 740A, 740B positioned at the distal ends of the fluid passageways 710, 720 can have cross-sectional areas that are greater than the cross-sectional areas of the openings 714, 724 between the passageways 710, 720 and the fluid chamber 706. Because of this the area of the filter that is active for trapping particulate matter can be larger than the areas of the openings 714, 724 between the passageways 710, 720 and the fluid chamber 706. In some implementations the flow of fluid through the filter 740, 740A, 740B can be reversed to dislodge some of the particulate matter that has been trapped by the filters from the filters.

The example pump devices 700 shown in FIGS. 7A, 7B, 7C include filters 740, 740A, 740B for blocking particulate matter in the fluid from entering a pump chamber of the device or for circulating in the fluidic system in which the pump devices operate. The filter 740 shown in FIG. 7A is disposed at the distal end 712 of the first fluid passageway 710, and the filter 740 shown in FIG. 7B is disposed at the distal end 722 of the second fluid passageway 720. These filters can include a plurality of openings in a foil, where the size of the openings is selected to block the passage of particles having a characteristic size greater than a threshold size and to allow fluid and particles having a characteristic size less than the threshold size to pass through the openings.

In some implementations, the example pump devices 700 shown in FIGS. 9A, 9B, 9C can include filters disposed within the first fluid passageway 710 or within the second fluid passageway 720, for example, between the first end 712 of the first fluid passageway 710 and the opening 714 at the second end of the first fluid passageway 710 and/or between the first end 722 of the second fluid passageway 720 and the opening 724 at the second end of the second fluid passageway 720. For example, as shown in FIG. 9A, the example pump device 700 can include a first filter 740C disposed within the first fluid passageway 710 and a second filter 740B disposed at the end of the second fluid passageway 720. In another example, as shown in FIG. 9B, the example pump device 700 can include a first filter 740A disposed at the end of the first fluid passageway 710 and a second filter 740C disposed within the second fluid passageway 720. In another example, as shown in FIG. 9C, the example pump device 700 can include filters 740C disposed within the first fluid passageway 710 and disposed within the second fluid passageway 720.

The filters 740C that are disposed within a fluid passageway 710, 720 can include an outer frame 750 that supports material within the frame that includes a plurality of small openings or passages through which fluid can pass but which have a threshold size that blocks particles having a characteristic size greater than the threshold size from passing through the filter 740C.

The outer frame 750 can be secured to the base plate 702 that defines the first fluid passageway 710. In some implementations, the base plate 702 can define a receptacle that receives the outer frame 750. In some implementations, the receptacle can have a lateral extent (e.g., a diameter) that is greater than the lateral extent of the first fluid passageway 710, such that when the outer frame 750 is disposed in the receptacle, an inner wall of the outer frame has a lateral extent that is similar to the lateral extent of the first fluid passageway 710. In some implementations, the outer frame can be press fit into the receptacle. In some implementations the outer frame 750 can be welded to the portion of the base plate 702 that defines the receptacle. In some implementations, after the outer frame 750 of the filter 740C is placed in the receptacle, a foil 742 can be placed over the outer frame 750 and then attached (e.g., welded) to the base plate 702.

In different implementations, the outer frame 750 can be made of different materials. For example, if the outer frame 750 is to be welded to a titanium base plate 702, the outer frame 750 can be made of titanium. In another example, if the outer frame 750 is to be securely press fit into a receptacle, the outer frame 750 can be made of a compliant material, for example, plastic, rubber, etc.

The material of the filter 740C supported by the outer frame 750, which includes a plurality of small openings or passages through fluid passes, can be made of different materials, which need not be identical or similar to the materials of the outer frame 750. For example, the material can include metal (e.g., titanium, gold, etc.). In another example the material can include ceramic material. In another example, the material can include plastic.

In some implementations, the thickness of the material of the filter, which includes the plurality of small openings or passages through which fluid passes, in the direction of the fluid flow through the filter can be greater than three times the mean lateral extent of the openings or passages through which the fluid passes. Thus, the openings or passages of the materials can operate more as tubes through which the fluid passes than as apertures in a thin plane of material. In some implementations, walls of the openings or passages of the material can be textured or treated to promote the adhesion of particulate matter, while also permitting the fluid to pass through the openings or passages. For example, the walls of the openings or passages can have a surface texture or roughness that facilitates the adhesion of particulate matter, and the surface of the openings or passages can include a hydrophobic coating to encourage the passage of fluid through the openings or passages.

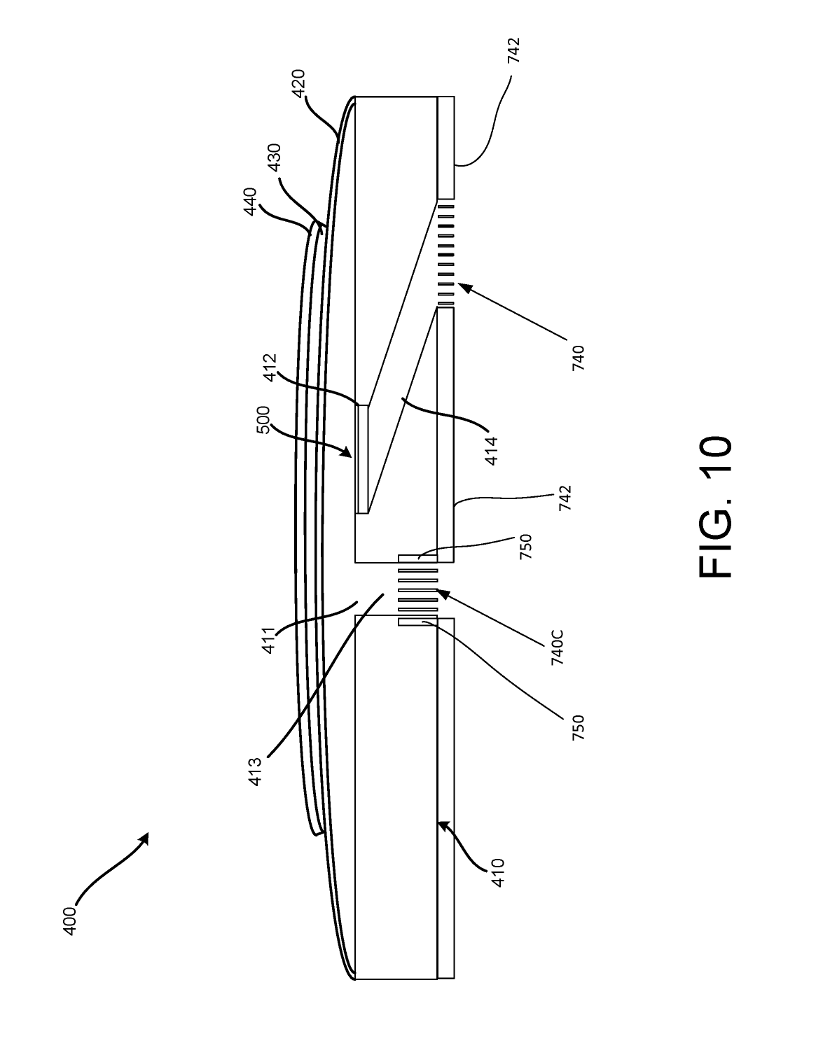

In addition to being used in the pumps described herein, the filters described herein also can be used in the valves described herein. For example, FIG. 10 is cross-sectional view of the valve device 400 shown in FIG. 5A and 5B, but also including a filter 740 located at an end of the second fluid passageway 414 and a filter 740C located within the first fluid passageway 413. The filters described herein also may be utilized in other valve structures described herein.

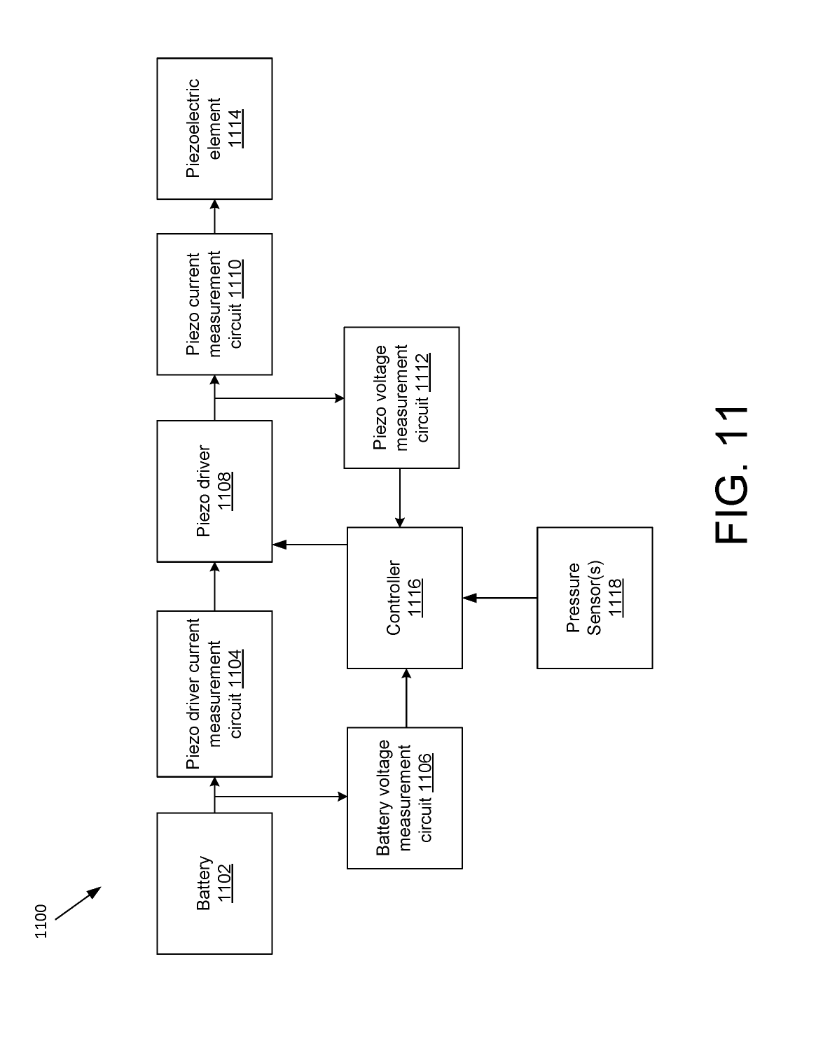

FIG. 11 is a schematic block diagram of an implantable fluid-operated system 1100 for driving a piezoelectric element 1114 of a piezoelectric-operated pump or valve and for monitoring and controlling the performance of the piezoelectric element. The system 1100 includes a battery 1102 that is configured to store electrical energy that can be used to drive the piezoelectric element 1114. A piezoelectric driver 1108 is electrically connected to the battery 1102 and to the piezoelectric element 1114. The piezoelectric driver 1108 includes electronic circuitry (e.g., analog and/or digital electronic circuitry) that is configured for receiving electrical energy from the battery 1102 and for generating a waveform of electrical energy that is provided to the piezoelectric element to drive the piezoelectric element.

In some implementations, the battery 1102 can provide electrical energy at a maximum voltage of 5 V or less, for example, at a maximum of 4.4 V or less to the piezoelectric driver 1108. The driver 1108 can step up the voltage and can output a waveform having a peak-to-peak voltage of greater than 50 V, for example, 100 V, to the piezoelectric element 1114. In some implementations, the driver 1108 can include step up transformer circuitry configured for receiving a first voltage signal from the battery 1102 and for outputting a second voltage signal to the piezoelectric element, where the second voltage is greater than the first voltage.

When the piezoelectric element 1114 is associated with a pump of the implantable inflatable device 100, 200, the driver 1108 can output a periodic waveform that is used to repeatedly change a volume of a fluid chamber to cause fluid to be pumped through the fluid chamber from one location to another, for example, from a reservoir to an inflatable member or from the inflatable member to the reservoir. In some implementations, the frequency of the periodic waveform can be between 30 Hz and 60 Hz, for example, 40-50 Hz. In some implementations, the periodic waveform can be a sine wave. In some implementations, the periodic waveform can include a series of square pulses. In some implementations, the periodic waveform can include a repeated series of waves provided to the piezoelectric element 1114, where the waves have a voltage that varies over time according to a function V=V(t) and where, unlike a sine wave, the second derivative of V divided by V (i.e., V’’(t)/V(t)) is not equal to one but where, unlike a square wave, V(t) does not include discontinuities, at which the first derivative of V(t) approaches infinity. When comparing two waveforms having an identical frequency and an identical peak-to-peak amplitude, a first waveform in the form of a sine wave may be more energy-efficient, in terms of preserving energy in the battery 1102, for driving the piezoelectric element 1114 than a second waveform in the form of a series of square pulses. More generally, a first waveform V₁(t) may be more energy-efficient, in terms of draining energy from the battery 1102, for driving the piezoelectric element 1114 to pump a certain volume of fluid than a second waveform V₂(t) when the maximum of V’’₁(t)/ V₁(t) is less than the maximum of V’’₂(t)/ V₂(t).

When the piezoelectric element 1114 is associated with a valve of the implantable inflatable device 100, 200, the driver 1108 can output a static voltage that is applied to the piezoelectric element of the valve to place the valve in an open or closed state. For example, in one implementation, a voltage of +88 V can be applied across the piezoelectric element to place the valve into a closed state, such that fluid is blocked from flowing through the valve, and a voltage of -12 V can be applied across piezoelectric element to place the valve in an open state, such that fluid can flow through the valve. When zero voltage is applied across the piezoelectric element, the valve may be in a partially open state, which can be a default state of the valve.

The system 1100 can include one or more monitor circuits configured for determining electrical parameters of the waveform that is provided by the driver 1108 to the piezoelectric element. For example, a current measurement circuit 1110 can measure an electric current drawn by the piezoelectric element 1114, and a voltage measurement circuit 1112 can measure a voltage of the waveform provided to the piezoelectric element 1114, while the piezoelectric element operates to pump fluid in the implantable device (in the case when piezoelectric element is associated with a pump) or while the piezoelectric element operates to maintain a state of a valve of the device (in the case when piezoelectric element is associated with a valve).

In addition, the system 1100 can include one or more monitor circuits configured for determining electrical parameters of electrical energy provided from the battery 1102 to the driver 1108. For example, a battery voltage measurement circuit 1106 can output a measured voltage of the battery 1102, and a battery current measurement circuit 1104 can measure a current drawn from the battery 1102 by the driver 1108 while the driver drives the piezoelectric element 1114 and/or powers other components of the system (e.g., a processor, a communication module, etc.).

The system 1100 also can include one or more pressure sensors 1118 that can measure parameters relevant to an operation of the system, such as, for example, a pressure of fluid at one or more locations of the system. For example, a first pressure sensor can be connected to a fluidic circuit between a piezoelectric pump and an inflatable member, where the pump supplies fluid from a reservoir and the inflatable member, to measure a fluid pressure in the inflatable member. In another example, a second pressure sensor can be connected to a fluidic circuit between the piezoelectric pump and a valve, where the pump supplies fluid from a reservoir to the inflatable member, and the valve is between the pump and the inflatable member and configured to measure a fluid pressure in the fluidic circuit between the pump and the valve. In another example, a third pressure sensor can be connected to a fluidic circuit between a reservoir and the piezoelectric pump to measure a fluid pressure in the reservoir.

A controller 1116 can receive signals indicating the parameters measured by the monitor circuits 1104, 1106, 1110, 1112 and can process the signals to diagnose the performance and status of the components of the system 1100. Based on the received signals, the controller 1116 may take action to change the performance of the system.

Referring again to FIG. 3, to maintain a positive fluid pressure in the inflatable member 204 after fluid has been pumped into the inflatable member from the reservoir 202, valves V1 and V2 both must be maintained in a closed state so that fluid does not flow from the inflatable member back into the reservoir. Referring to FIG. 4C, to maintain the valves V1 and V2 in their closed states, a sufficient force must be exerted on the diaphragm 420, such that the diaphragm is pressed against the O-ring seal 450 and that fluid does not flow past the seal from the second fluid passageway 414 to the first fluid passageway 413. Because the force exerted by the piezoelectric element 440 on the diaphragm 420 depends on the voltage across the piezoelectric element, a sufficient voltage must be maintained on the piezoelectric element 440 of the valve in order to maintain the valve in its closed state, such that fluid does not flow past the seal 450.

However, in a real piezoelectric valve used in an implantable fluid-operated inflatable device, non-negligible leakage currents can exist, which can cause the voltage on a piezoelectric valve to decay and the valve to begin to leak. For example, when a piezoelectric driver is coupled to the piezoelectric element of a valve, charge can leak from the piezoelectric valve through the piezoelectric driver to ground, thus resulting in a leakage current that can decrease the voltage on the piezoelectric valve and therefore a reduction in the force with which the valve is held in its closed state. After enough charge has leaked from the piezoelectric valve, fluid can begin to leak past a seal of the valve, thereby causing a fluid pressure in the inflatable member to decrease. With typical leakage currents, the voltage on a piezoelectric valve can decay over a timescale that is shorter than a typical timescale over which a valve should remain closed, which can cause performance problems for an implantable fluid-operated inflatable device or which could require that electric charge be continually, or periodically, added to piezoelectric valve to maintain a predetermined voltage state of the piezoelectric valve, thereby decreasing the battery lifetime of the device.

Therefore, to mitigate the effects of such leakage currents, techniques described herein can isolate the piezoelectric valve from significant pathways of leakage current and/or can reduce the amount of time that the piezoelectric valve is connected to a significant pathway of leakage current.

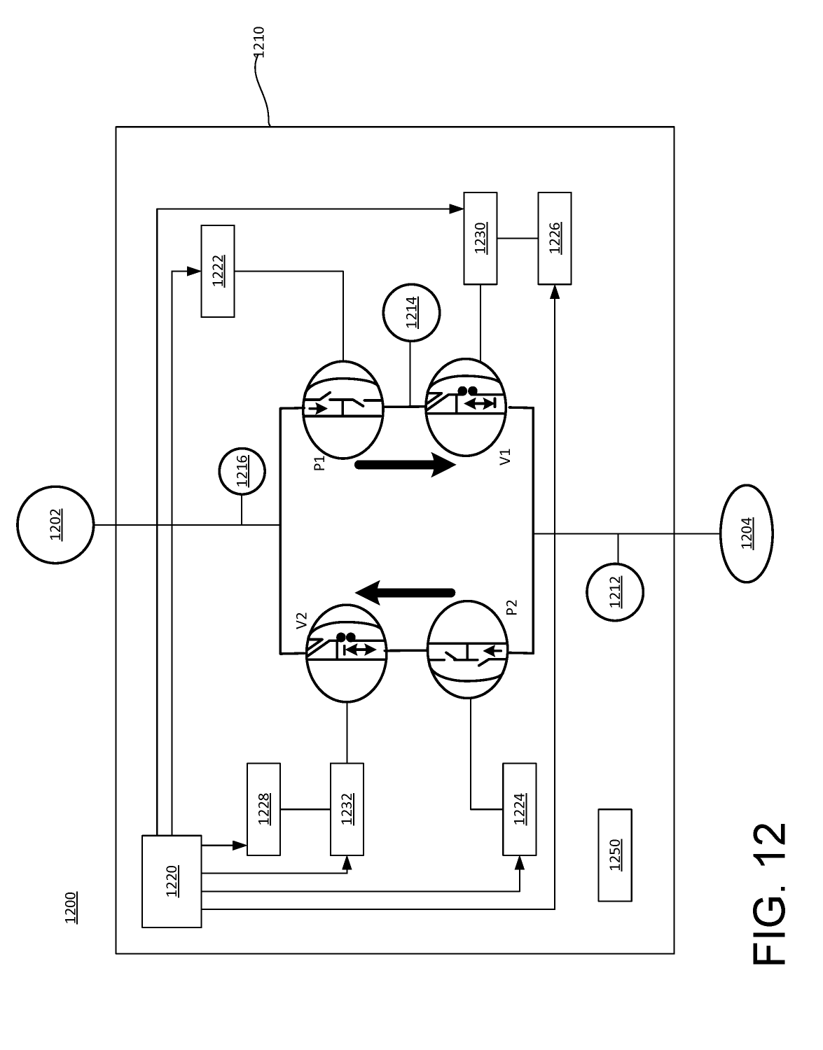

FIG. 12 is a schematic diagram of an implantable fluid-operated inflatable device 1200 that reduces leakage current from piezoelectric valves of the device. The device 1200 can include a fluid reservoir 1202, an inflatable member, and a housing 1210 that includes a number of components that transfer fluid between the reservoir 1202 in the inflatable member 1204.

For example, a first pump P1 and a first valve V1 can be positioned in a first fluid passageway, between the reservoir 1202 and the inflatable member 1204, to control the flow of fluid from the reservoir 1202 to the inflatable member 1204, and a second pump P2 and a second valve V2 can be positioned in a second fluid passageway, between the inflatable member 1204 and the reservoir 1202, to control the flow of fluid from the inflatable member 1204 to the reservoir 1202. The first pump P1 and the first valve V1 operate to pump fluid from the reservoir 1202 to the inflatable member 1204 through the first fluid passageway to provide for inflation of the inflatable member 1204, while the second valve V2 closes the second fluid passageway to prevent backflow of fluid, back to the reservoir 1202. The second pump P2 and the second valve V2 operate to pump fluid from the inflatable member 1204 to the reservoir 1202 through the second fluid passageway to provide for deflation of the inflatable member 1204, while the first valve V1 closes the first fluid passageway to prevent backflow of fluid to the inflatable member 1204.

In some implementations, the example fluidic architecture can include one or more pressure sensors 1212, 1214, 1216, each configured to measure a fluid pressure at a point in the system. For example, a first pressure sensor 1212 can be connected to a fluidic passageway, conduit, chamber or component located fluidically between the inflatable member 1204 and pumps P1, P2 and valves V1, V2, and can be configured to measure a fluid pressure at this location, which can also serve as a measure of a fluid pressure in the inflatable member(s) 1204, because the fluid is essentially incompressible and the conduit between the pressure sensor 1212 and the inflatable member(s) 1204 can be considered to be free of obstruction. A second pressure sensor 1214 can be connected to a fluidic passageway, conduit, chamber or component located fluidically between pump P1 and valve V1 and can be configured to measure a fluid pressure at this location. A third pressure sensor 1216 can be connected to a fluidic passageway, conduit, chamber or component located fluidically between the reservoir 1202 and pumps P1, P2 and valves V1, V2, and can be configured to measure a fluid pressure at this location, which can also serve as a measure of a fluid pressure in the reservoir, because the fluid is essentially incompressible and the conduit between the pressure sensor 1216 and the reservoir 1202 can be considered to be free of obstruction.

The controller 1220 (e.g., a microprocessor, an ASIC, etc.) can control one or more drivers that control the supply of electrical signals to the pumps and valves of the device 1200. For example, the controller 1220 can control a pump driver 1222 that provides a time-varying electrical waveform to pump P1 to drive the operation of pump P1 and can control a pump driver 1224 that provides a time-varying electrical waveform to pump P2 to drive the operation of pump P2. The controller 1220 can control a valve driver 1226 that provides a DC voltage to valve V1 to drive the operation of valve V1 and can control a valve driver 1228 that provides a DC voltage to valve V2 to drive the operation of valve V2. Energy for the electrical signals can be supplied by an energy storage device 1250.

The valve driver 1226 can be connected to the valve V1 by a high impedance switch 1230, and the switch can be controlled by the controller 1220. The controller 1220 can control the switch 1230 to be in a closed state to connect the valve driver 1226 to the piezoelectric valve V1, to change or measure a voltage on the valve, and can control the switch 1230 to be in an open state to disconnect the valve driver 1226 from the piezoelectric valve V1, to reduce a leakage current from the piezoelectric valve, so that a voltage on the piezoelectric valve can be maintained. Similarly, the valve driver 1228 can be connected to the valve V2 by a high impedance switch 1232, and the switch can be controlled by the controller 1220. The controller 1220 can control the switch 1232 to be in a closed state to connect the valve driver 1228 to the piezoelectric valve V2, to change or measure a voltage across the piezoelectric valve, and can control the switch 1232 to be in an open state to disconnect the valve driver 1228 from the piezoelectric valve V2, to reduce a leakage current from the piezoelectric valve, so that a voltage on the piezoelectric valve can be maintained.

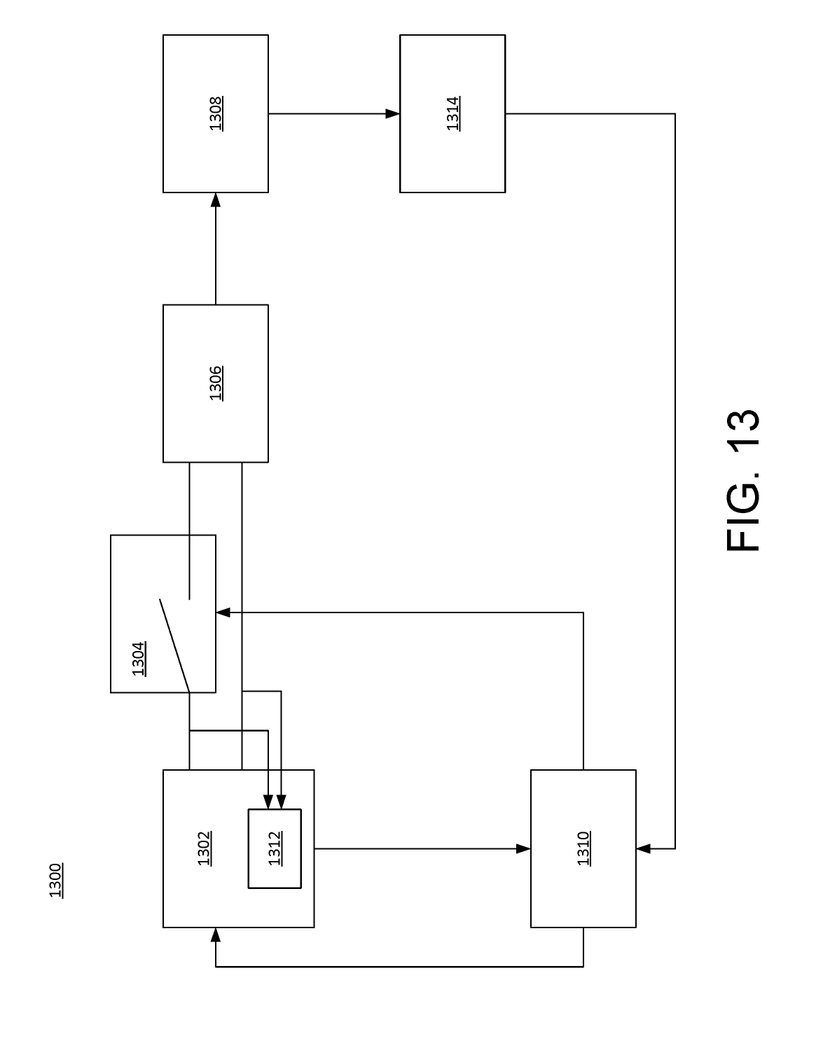

FIG. 13 is a schematic diagram of a system 1300 including components of an implantable fluid-operated inflatable device used to operate a piezoelectric valve of the device. The system 1300 includes a valve driver 1302 and a piezoelectric valve 1306, whose operation is controlled by the valve driver. For example, the valve driver 1302 can supply a DC voltage signal to the piezoelectric valve 1306 to control the valve between an open state and a closed state. The state of the piezoelectric valve 1306 can affect an operation of a fluidic system 1308 of the implantable fluid-operated inflatable device. For example, when the piezoelectric valve 1306 is in its closed state, fluid can be blocked from flowing in one or more portions of the fluidic system 1308, and when the piezoelectric valve is in its open state, fluid can be free to flow in the one or more portions of the fluidic system.

The valve driver 1302 can be connected to the piezoelectric valve 1306 by way of a high impedance switch 1304. In some implementations, the switch 1304 includes a solid state relay. In some implementations, the switch 1304 can include a bidirectional, multi-FET circuit. In some implementations, the switch 1304 can include a Reed switch. When the switch 1304 is closed, the valve driver 1302 can be connected to the piezoelectric valve 1306, and when the switch 1304 is open, the valve driver 1302 can be disconnected from the piezoelectric valve 1306. A controller 1310 (e.g., a processor, an ASIC, etc.) can be electrically coupled to the valve driver 1302 and to the switch 1304, to control the operation of the valve driver and the switch 1304. A leakage current through the switch 1304 can be, for example, less than 30 nA, less than 10 nA, or less than 3nA when the switch is in the open state. In particular, the leakage current through the switch can be small enough, so that the piezoelectric valve 1306 remains closed for a period of time greater than a threshold amount of time, even when the switch 1304 is open and the valve 1306 is not supplied with charge by the valve driver 1302. For example, when the threshold amount of time is hour hours, if the piezoelectric valve has a capacitance of 1.54 microfarads and is charged to 88 V, where a minimum charge of 60 V on the valve is required to maintain the valve in a closed state, then a switch having a leakage current of 3 nA may maintain the valve in a closed state for 4.0 hours, according to the equation, t = C*ΔV/I, where C is the capacitance of the valve, ΔV is the change of voltage on the valve, and I is the leakage current from the valve.

In some implementations, the valve driver 1302 can include a voltage sensing circuit 1312 that measures a voltage across the piezoelectric valve 1306. In some implementations, the switch 1304 must be closed for the valve driver 1302 to be coupled to the piezoelectric valve 1306, so that the voltage sensing circuit 1312 can measure the voltage across the piezoelectric valve 1306. The controller 1310 can control the valve driver 1302 and the switch 1304 based on the voltage measured across the piezoelectric valve 1306.

The system 1300 can include one or more pressure sensors 1314 that are fluidically-coupled to the fluidic system 1308 to measure a pressure in the fluidic system 1308. The controller 1310 can receive a signal from the one or more pressure sensors 1314 indicating a pressure in the fluidic system 1308, and the controller can control the valve driver 1302 and the switch 1304 based on the indication of the pressure in the fluidic system 1308.

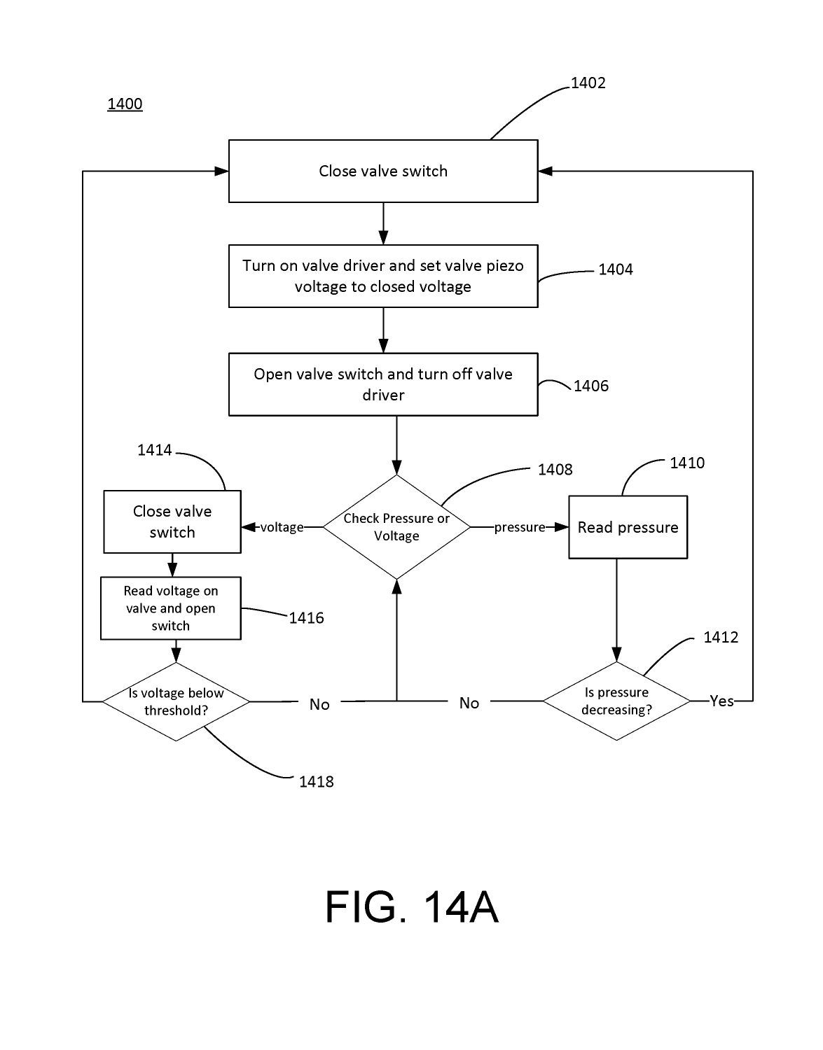

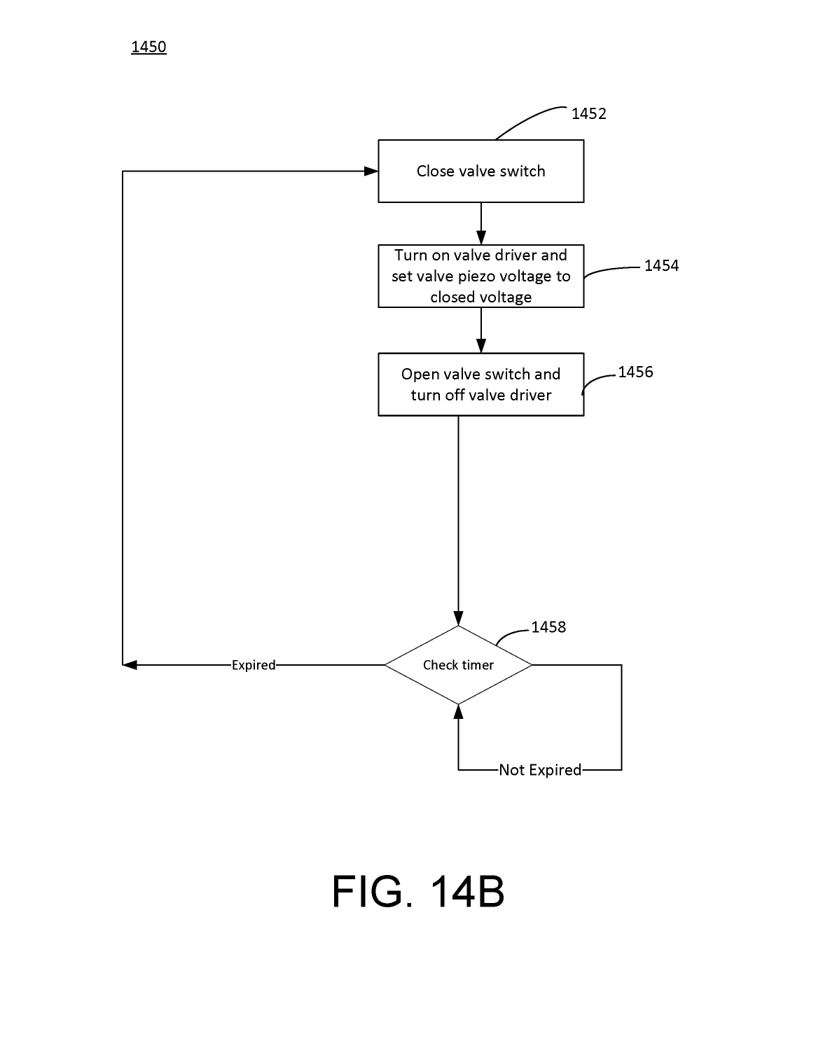

FIG. 14A is a schematic diagram of a process 1400 for controlling the system 1300 to close the piezoelectric valve and to maintain the piezoelectric valve in its closed state. In the process 1400, the controller 1310 sends a signal to the switch 1304 to close the switch (1402). The controller 1310 sends a signal to the valve driver 1302 to turn on the valve driver and to operate the driver to provide a predetermined voltage across the piezoelectric valve 1306, where the predetermined voltage is sufficient to close the valve (1404). The voltage sensing circuit 1312 of the valve driver 1302 monitors the voltage across the piezoelectric valve 1306 and supplies charge to the piezoelectric valve until the voltage across the piezoelectric valve reaches the predetermined voltage. In some implementations, the valve driver 1302 supplies charge to the piezoelectric valve for a period of time after the predetermined voltage is reached, for example, to saturate the material of the piezoelectric element of the valve 1306. For example, the valve driver 1302 can supply charge to the piezoelectric valve 1306 for at least two seconds.

Once the voltage has reached the predetermined voltage, the controller 1310 can send a signal to the switch 1304 to open the switch and can send a signal to the valve driver 1302 to cease the provision of charge from the valve driver to the piezoelectric valve (1406). In the state, because of the high impedance of the switch 1304, charge can be maintained across the piezoelectric valve 1306, such that the piezoelectric valve remains in its closed state.

Thereafter, the pressure of the fluidic system associated with the piezoelectric valve 1306 can be checked or the voltage on the piezoelectric valve can be checked (1408) to ensure that the valve 1306 remains closed and/or that fluid has not leaked from the fluidic system 1308 (e.g., an inflatable member). If the pressure is checked, a pressure in the fluidic system can be measured (1410), and if the pressure is below a threshold pressure (1412), the pressure of the fluidic system can be increased above the threshold pressure (e.g., by operating a piezoelectric pump to re-inflate an inflatable cylinder), and the controller 1310 then can close the valve switch (1402) to begin the process of closing the piezoelectric valve 1306. If the pressure is above the threshold pressure (1412), the pressure or voltage can be checked at a later time (1408).

If the voltage is checked (1408), the controller 1310 can send a signal to the high impedance switch 1304 to close the switch (1414) and connect the valve driver 1302 to the piezoelectric valve 1306. The controller 1310 can send a first signal to the valve driver 1302 to measure a voltage across the piezoelectric valve and can send a second signal to the switch to open the switch after the measurement is complete (1416). If the measured voltage is less than a threshold voltage (1418), the controller 1310 then can close the valve switch (1402) to begin the process of closing the piezoelectric valve 1306 with the application of a voltage across the piezoelectric valve that exceeds the predetermined voltage. If the measured voltage is above the threshold voltage (1418), the pressure or voltage can be checked at a later time (1408). As used herein, if the threshold voltage is positive, then the measured voltage is less than the threshold voltage if the measured voltage has a lower value than the threshold voltage, and if the threshold voltage is negative, then the measured voltage is less than the threshold voltage if the measured voltage has a higher value (e.g., is less negative) than the threshold voltage.

By measuring the voltage across the piezoelectric valve, it may be possible to predict that the fluidic system 1308 will begin to leak, even when the pressure in the fluidic system exceeds the predetermined pressure and the system is not currently leaking. For example, if the voltage across the piezoelectric element has decayed from a predetermined value below a threshold value, where the threshold value is nevertheless sufficient to maintain the piezoelectric valve in a closed state, the decay of the voltage from its predetermined value may indicate that the piezoelectric valve will begin to leak when the voltage continues to decay. Therefore, by taking action to close the valve switch (1402) and to add charge to the piezoelectric valve, the piezoelectric valve can be maintained in its close state without fluid leaking from the system.

FIG. 14B is a schematic diagram of another process 1450 for controlling the system 1300 to close the piezoelectric valve and to maintain the piezoelectric valve in its closed state. In the process 1450, the controller 1310 sends a signal to the switch 1304 to close the switch (1452). The controller 1310 sends a signal to the valve driver 1302 to turn on the valve driver and to operate the driver to provide a predetermined voltage across the piezoelectric valve 1306, where the predetermined voltage is sufficient to close the valve (1454). The voltage sensing circuit 1312 of the valve driver 1302 monitors the voltage across the piezoelectric valve 1306 and supplies charge to the piezoelectric valve until the voltage across the piezoelectric valve reaches the predetermined voltage. In some implementations, the valve driver 1302 supplies charge to the piezoelectric valve for a period of time after the predetermined voltage is reached, for example, to saturate the material of the piezoelectric element of the valve 1306. For example, the valve driver 1302 can supply charge to the piezoelectric valve 1306 for at least two seconds.

Once the voltage has reached the predetermined voltage, the controller 1310 can send a signal to the switch 1304 to open the switch and can send a signal to the valve driver 1302 to cease the provision of charge from the valve driver to the piezoelectric valve (1456). In the state, because of the high impedance of the switch 1304, charge can be maintained across the piezoelectric valve 1306, such that the piezoelectric valve remains in its closed state.

Thereafter, charge can be added to the piezoelectric element of the piezoelectric valve 1306 periodically to maintain the valve in a closed state. For example, a current leakage from the piezoelectric valve 1306 can be predetermined (e.g., it may be determined empirically from measurements of the voltage on the piezoelectric valve over time, or it may be specified by a manufacturer of the valve), and charge can be added to the valve 1306 periodically to replenish the charge that is determined to leak from the valve. In one implementation, a timer can be started and checked to determine if it has reached a threshold time (1458). If the timer has not reached the threshold time, the timer can be checked at a later time, but when the timer reaches the threshold time, it can be assumed that the voltage on the piezoelectric valve 1306 likely is below a threshold voltage, and therefore the controller 1310 can again operate to begin the process 1450 to close the switch (1452), turn on the valve driver and set voltage across the piezoelectric valve to the predetermined voltage to close the valve (1454), and then to open the switch (1456).

By periodically adding charge to the piezoelectric valve 1306, voltage across the valve can maintained above a threshold voltage, despite the leakage of charge from the valve. Therefore, by taking action to periodically close the valve switch (1452), turn on the valve driver to add charge to the valve, the piezoelectric valve 1306 can be maintained in its close state without fluid leaking from the system.

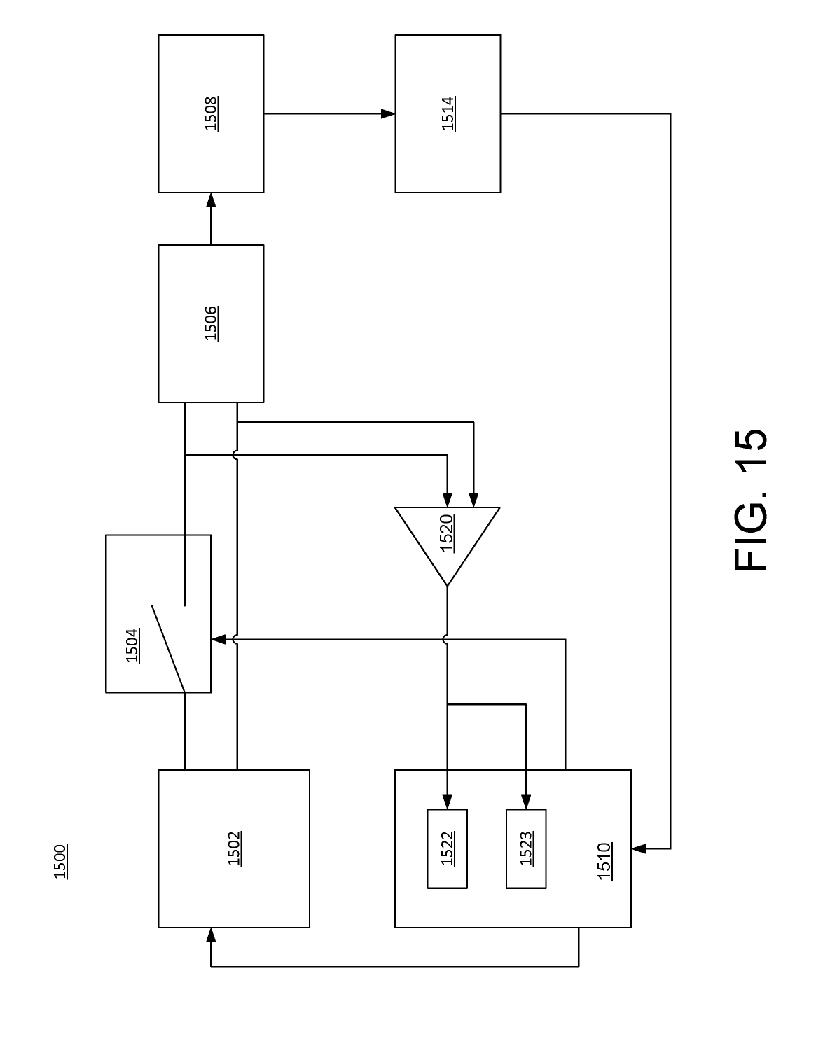

FIG. 15 is a schematic diagram of another system 1500 including components of an implantable fluid-operated inflatable device used to operate a piezoelectric valve of the device. Like the system 1300, the system 1500 reduces leakage current from a piezoelectric valve of the system.

The system 1500 includes a valve driver 1502 and a piezoelectric valve 1506, whose operation is controlled by the valve driver. For example, the valve driver 1502 can supply a DC voltage signal to the piezoelectric valve 1506 to control the valve between an open state and a closed state. The state of the piezoelectric valve 1506 can affect an operation of a fluidic system 1508 of the implantable fluid-operated inflatable device. For example, when the piezoelectric valve 1506 is in its closed state, fluid can be blocked from flowing in one or more portions of the fluidic system 1508, and when the piezoelectric valve is in its open state, fluid can be free to flow in the one or more portions of the fluidic system.

The valve driver 1502 can be connected to the piezoelectric valve 1506 by way of a high impedance switch 1504. In some implementations, the switch 1504 includes a solid-state relay. In some implementations, the switch 1504 can include a bidirectional, multi-FET circuit. In some implementations, the switch 1504 can include a Reed switch. When the switch 1504 is closed, the valve driver 1502 can be connected to the piezoelectric valve 1506, and when the switch 1504 is open, the valve driver 1502 can be disconnected from the piezoelectric valve 1506. A controller 1510 (e.g., a processor, an ASIC, etc.) can be electrically coupled to the valve driver 1502 and to the switch 1504, to control the operation of the valve driver and the switch 1504.

The system 1500 can include one or more pressure sensors 1514 that are fluidically-coupled to the fluidic system 1508 to measure a pressure in the fluidic system 1508. The controller 1510 can receive a signal from the one or more pressure sensors 1514 indicating a pressure in the fluidic system 1508, and the controller can control the valve driver 1502 and the switch 1504 based on the indication of the pressure in the fluidic system 1508.

In some implementations, the system can include an isolation circuit 1520, for example, a buffer or an instrumentation amplifier, which has a high input impedance and a low output impedance, between the piezoelectric valve 1506 and the controller 1510 that can isolate the voltage on the piezoelectric valve from a circuit that measures the voltage. The isolation circuit 1520 can be connected between the controller 1510 on a first side and the piezoelectric valve 1506 on a second side, with the second side being connected to the piezoelectric valve between the valve and the switch 1504. Therefore, the switch need not be closed to measure a voltage across the piezoelectric valve, which reduces an amount of charge leakage from the piezoelectric valve when a voltage measurement is performed. An output of the isolation circuit 1520 can be provided to an analog to digital converter (ADC) 1522 within the controller 1510, where an output from the ADC provides a measurement of the voltage across the piezoelectric valve 1506, and the output can be proportional to the measurement of the voltage across the piezoelectric valve 1506. Because the ADC 1522 of the controller 1510 can be used to measure the voltage across the piezoelectric valve based on an analog voltage taken from the valve side of the switch 1504, the switch need not be closed to make the measurement, and steps (1414) and (1416) of process 1400 shown in FIG. 14A can be eliminated from the process 1400 when the isolation circuit 1520 of FIG. 15 is used. Thus, the voltage can be measured continuously, without closing and opening the switch 1504.

Referring again to FIG. 15, the input impedance of the isolation circuit 1520 can be, for example, greater than 1 GΩ, greater than 3 GΩ, or greater than 10 GΩ. In particular, the input impedance can be great enough, so that the piezoelectric valve 1506 remains closed for a period of time greater than a threshold amount of time, even when the isolation circuit 1520 is connected between the piezoelectric valve 1506 and the controller 1510 and the valve 1506 is not supplied with charge by the valve driver 1502. For example, when the threshold amount of time is four hours, if the piezoelectric valve has a capacitance of 1.54 microfarads and is charged to voltage of 88 V, where a voltage of 60 V on the valve is required to maintain the valve in a closed state, then an isolation circuit 1520 having an input impedance of 24.4 GΩ may maintain the valve in a closed state for 4.0 hours, according to the equation, t = -(RC)*ln(V_{f}/V₀), where C is the capacitance of the valve, R is the input impedance, V_{f} is the minimum voltage on the valve, and Vₒ is the initial voltage on the valve.

Referring again to FIG. 13, closing and opening the switch 1304 to measure the voltage across the piezoelectric valve 1306 can require recharging the piezoelectric valve after the voltage measurement is made to replenish charge lost to leakage current during the measurement process. However, it is possible that repeatedly changing a voltage applied to piezoelectric valve, as a result of measuring the voltage with the system of FIG. 13 could affect the polarization of the piezoelectric material of the valve 1306, thereby reducing the mechanical response of the piezoelectric material to a voltage applied to the piezoelectric material. Thus, when the isolation circuit 1520 of FIG. 15 is used, and the switch 1504 is not closed and opened to measure a voltage across the piezoelectric valve, the polarization of the piezoelectric material may be relatively unaffected, thereby preserving a mechanical response of the piezoelectric material to a voltage applied to the piezoelectric material.

In some implementations, the isolation circuit 1520 can include, for example, a transformer, an opto-coupler, or a digital isolator configured to transfer a signal indicating that voltage across the piezoelectric valve 1506 has dropped below a threshold value, while isolating the piezoelectric valve 1506 from the controller 1510 to maintain a very low leakage current from the valve 1506. The isolation circuit 1520 also may include a comparator that compares a voltage on the piezoelectric valve to a reference voltage, and where the output of the comparator changes state from HI to LO (or from LO to HI), when the voltage on the piezoelectric valve drops below the reference voltage and the output from the comparator can be processed by the controller 1510 to determined when to add charge to the piezoelectric valve to maintain the valve in a closed state. In such implementations, the signal indicating that voltage across the piezoelectric valve 1506 has dropped below a threshold value can be processed by the ADC 1522 of the controller 1510 or by an alternative logic circuit 1523, such that the controller 1510 can respond to an indication that a voltage across the piezoelectric valve 1506 has dropped below a threshold value by causing switch 1504 to close and the driver 1502 to add charge to the valve 1506.

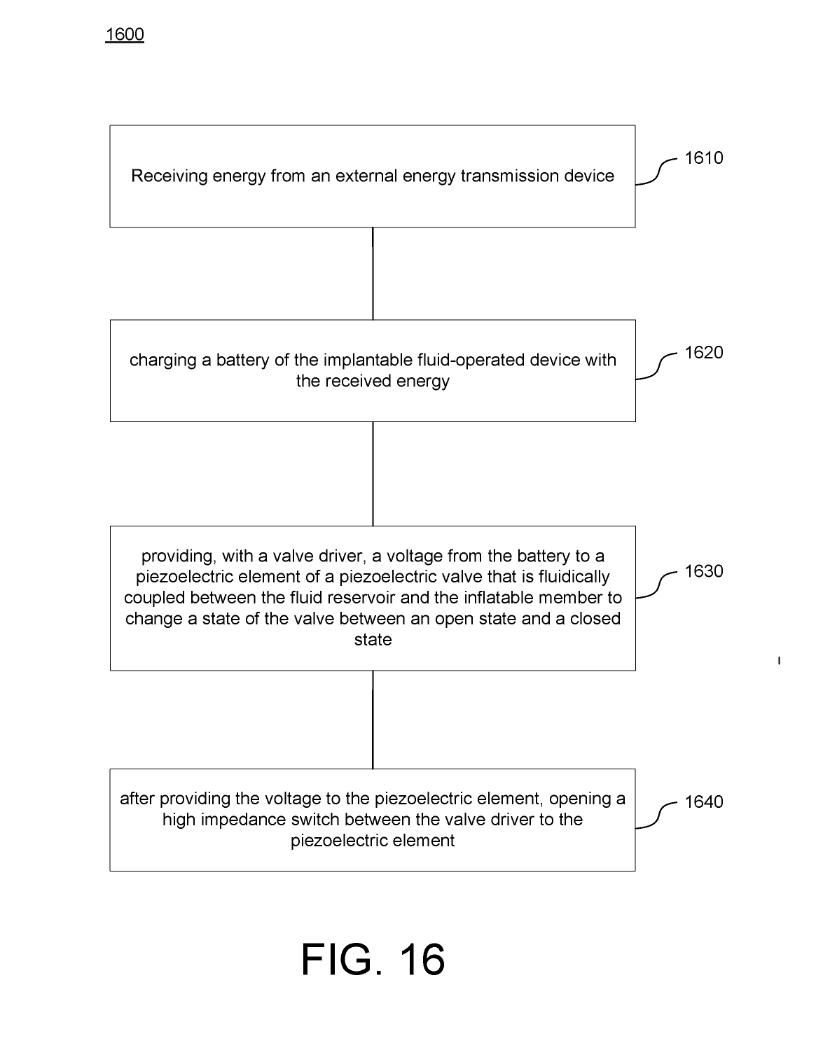

FIG. 16 is a flowchart of an example process 1600 for controlling fluid flow between a fluid reservoir and an inflatable member of an implantable fluid-operated device. The process 1600 includes, at step 1610, receiving energy from an external energy transmission device. The process 1600 includes, at step 1620, charging a battery of the implantable fluid-operated device with the received energy. The process 1600 further includes, at step 1630, providing, with a valve driver, a voltage from the battery to a piezoelectric element of a piezoelectric valve that is fluidically coupled between the fluid reservoir and the inflatable member to change a state of the valve between an open state and a closed state. The process 1600 further includes, at step 1640, after providing the voltage to the piezoelectric element, opening a high impedance switch between the valve driver to the piezoelectric element.

While certain features of the described implementations have been illustrated as described herein, many modifications, substitutions, changes and equivalents will occur to those skilled in the art. It is, therefore, to be understood that the appended claims are intended to cover all such modifications and changes as fall within the scope of the embodiments.

What is claimed is:

1. An implantable fluid-operated device configured to control fluid flow between a fluid reservoir and an inflatable member, the device comprising:
a battery configured for storing energy;
energy transmission circuitry configured for receiving energy from an external energy transmission device and providing energy to charge the battery;
a valve including: a base plate, a deformable diaphragm, a piezoelectric element coupled to the deformable diaphragm, and a fluid chamber defined between the base plate and the deformable diaphragm;
a valve driver configured for providing a voltage to the piezoelectric element to change a state of the valve between an open state and a closed state; and
a high impedance switch configured for switching the voltage from the valve driver to the piezoelectric element.

2. The implantable fluid-operated device of claim 1, wherein the high impedance switch includes a solid state relay.

3. The implantable fluid-operated device of claim 1, wherein the high impedance switch includes a bidirectional, multi-FET circuit.

4. The implantable fluid-operated device of claim 1, wherein the valve driver includes voltage sensing circuitry configured for measuring a voltage across the piezoelectric element when the high impedance switch is connected between the valve driver and the valve.

5. The implantable fluid-operated device of claim 1, wherein the inflatable member includes an inflatable cuff configured for implantation about a urethra of a patient.

6. The implantable fluid-operated device of claim 1, wherein the inflatable member includes a penile implant.

7. The implantable fluid-operated device of claim 1, further comprising a controller, the controller being configured to:
close the switch;
cause the valve driver to provide a predetermined voltage to the piezoelectric element when the switch is closed;
open the switch;
cause the valve driver to measure a voltage on the piezoelectric element when the switch is closed; and
cause the valve driver to provide the predetermined voltage to the piezoelectric element when the measured voltage is less than a threshold voltage.

8. The implantable fluid-operated device of claim 1, further comprising a controller, the controller being configured to:
close the switch;
cause the valve driver to provide a predetermined voltage to the piezoelectric element when the switch is closed;
open the switch;
repeatedly, when the switch is open, determine whether a time after the opening of the switch exceeds a threshold time; and
when the time exceeds the threshold time:
close the switch;
cause the valve driver to add charge to the piezoelectric element to provide the predetermined voltage to the piezoelectric element; and
open the switch.

9. The implantable fluid-operated device of claim 1, further comprising:
a controller; and
an isolation circuit connected between the controller and the piezoelectric element and being configured to providing a voltage signal proportional to a voltage across the piezoelectric element,
wherein the controller configured to:
close the switch;
cause the valve driver to provide a predetermined voltage to the piezoelectric element when the switch is closed;
open the switch;
when the switch is open, determine a voltage across the piezoelectric element based on the voltage signal; and
cause the valve driver to provide the predetermined voltage to the piezoelectric element when the determined voltage is less than a threshold voltage.

10. The implantable fluid-operated device of claim 9, wherein the isolation circuit includes a buffer.

11. The implantable fluid-operated device of claim 9, wherein the isolation circuit includes an instrumentation amplifier.

12. The implantable fluid-operated device of claim 1, further comprising:
a pressure sensor configured for measuring a pressure in the inflatable member; and
a controller configured to, in response to a pressure measured by the pressure sensor being less than a threshold pressure:
close the switch; and
cause the valve driver to provide a predetermined voltage to the piezoelectric element.

13. A method of controlling fluid flow between a fluid reservoir and an inflatable member of an implantable fluid-operated device, the method comprising:
receiving energy from an external energy transmission device;
charging a battery of the implantable fluid-operated device with the received energy;
providing, with a valve driver, a voltage from the battery to a piezoelectric element of a piezoelectric valve that is fluidically coupled between the fluid reservoir and the inflatable member to change a state of the valve between an open state and a closed state;
after providing the voltage to the piezoelectric element, opening a high impedance switch between the valve driver to the piezoelectric element.

14. The method of claim 13, wherein the inflatable member includes an inflatable cuff configured for implantation about a urethra of a patient.

15. The method of claim 13, wherein the inflatable member includes a penile implant.

16. The method of claim 13, further comprising:
closing the switch before the voltage is provided to the piezoelectric element;
providing, with the valve driver, a predetermined voltage to the piezoelectric element when the switch is closed;
after providing the predetermined voltage to the piezoelectric element, opening the switch;
when the switch is open, repeatedly determining whether a time after the opening of the switch exceeds a threshold time; and
when the time exceeds the threshold time:
closing the switch;
adding charge from the battery to the piezoelectric element to provide the predetermined voltage to the piezoelectric element; and
opening the switch.

17. The method of claim 13, further comprising:
generating, with an isolation circuit that is connected between a controller and the piezoelectric element, a voltage signal proportional to a voltage across the piezoelectric element;
when the switch is open, determining a voltage across the piezoelectric element based on the voltage signal;
providing a predetermined voltage to the piezoelectric element when the determined voltage is less than a threshold voltage.

18. The method of claim 17, wherein the isolation circuit includes a buffer.

19. The method of claim 17, wherein the isolation circuit includes an instrumentation amplifier.

20. The method of claim 13, further comprising:
measuring a pressure in the inflatable member; and
in response to the measured pressure being less than a threshold pressure:
closing the switch; and
providing a predetermined voltage to the piezoelectric element.
